(19) United States
(12) Patent Application Publication
(10) Pub. No.: US 2007/0154399 A1
(43) Pub. Date: Jul. 5, 2007

(54) Immunotherapy for immune suppressed patients

(76) Inventor: John W. Hadden, Cold Spring Harbor, NY (US)

Correspondence Address: Kenneth L. Kohn;KOHN & ASSOCIATES, PLLC, Suite 410, 30500 Northwestern Highway, Farmington Hills, MI, 48337, US

(21) Appl. No.: 11/706,946

(22) Filed: Feb. 15, 2007

Related U.S. Application Data
(63) Continuation-in-part of application No. 11/374,783, filed on Mar. 14, 2006.
Application No. 11/374,783 is a continuation-in-part of application No. 10/637,869, filed on Aug. 8, 2003, now Pat. No. 7,182,942.
Application No. 10/637,869 is a continuation-in-part of application No. 10/015,123, filed on Oct. 26, 2001, now Pat. No. 6,977,072.
(60) Provisional application No. 60/243,912, filed on Oct. 27, 2000.

(51) Int. Cl. A61K 49/00; A61K 38/19; A61K 38/20; A61K 38/21

(52) U.S. Cl. 424/9.81; 424/85.1; 424/85.2; 424/85.6

(57) ABSTRACT

A diagnostic skin test for predicting treatment outcome, consisting essentially of an effective amount of an NCM or a T lymphocyte mitogen of muromonab-CD3. A kit for performing a skin test consisting essentially of an effective amount of an NCM or a T lymphocyte mitogen of muromonab-CD3. A method of performing a skin test on a patient, consisting essentially of the steps of administering an effective amount of an NCM or a T lymphocyte mitogen of muromonab-CD3 to skin, analyzing results of the skin test, and predicting a treatment outcome. Methods of detecting defects in monocyte or T lymphocyte function, including the steps of administering an effective amount of an NCM or T lymphocyte mitogen of muromonab-CD3 to skin, analyzing results of the skin test, and detecting at least one defect in monocyte or T lymphocyte function. A mechanism for indicating a functioning efferent or afferent limb of an immune system, including a diagnostic skin test including an effective amount of an NCM or a T lymphocyte mitogen of muromonab-CD3.

### CROSS-REFERENCE TO RELATED APPLICATIONS

This application is a continuation-in-part of U.S. patent application Ser. No. 11/374,783, filed Mar. 14, 2006, which is a continuation-in-part of U.S. patent application Ser. No. 10/637,869, filed Aug. 8, 2003, which is a continuation-in-part of U.S. patent application Ser. No. 10/015,123 (now U.S. Pat. No. 6,977,072), filed Oct. 26, 2001, which claims the benefit of priority under 35 U.S.C. Section 119(e) of U.S. Provisional Patent Application No. 60/243,912, filed Oct. 27, 2000, all of which are incorporated herein by reference.

### BACKGROUND OF THE INVENTION

1. Technical Field

The present invention relates to the field of personalized medicine, and more specifically, to a diagnostic skin test for the detection of treatment outcomes.

2. Background Art

Cellular immunodeficiency is a deficiency of immune response in which the body is not able to effectively protect itself from harmful antigens. The immune system in this condition is effectively turned down or completely turned off. Such deficiency can be drug-induced, e.g., by drug treatment; virus-induced, e.g., as in AIDS; or disease-induced, e.g., by cancer. In fact, cellular immunodeficiency is common among cancer patients. The body is not able to detect, and thus protect against, tumor antigens, allowing a tumor to grow and possibly metastasize.

Cellular immunodeficiency, whether cancer related or not, can be due to several different causes such as T cell, dendritic cell (DC), and/or monocyte functional defects. While T lymphocytopenia is believed to be due to T cell functional defects, other cellular immunodeficiencies can be traced to one or more monocyte or dendritic cell functional defects. Monocytes as defined herein are essentially synonymous with adherent peripheral blood mononuclear cells (PBMCs) and are precursors to myeloid-derived macrophages and dendritic cells.

Defects in monocyte function can have wide-ranging effects on immune function. For example, because monocytes and macrophages play an important role in the generation of cell-mediated immunity and inflammation, monocyte functional defects may correlate with negative or reduced cell-mediated immune responses such as those detected by standard cell mediated immunity (CMI), also called delayed type hypersensitivity (DTH).

The impaired function of dendritic cells in cancer-bearing hosts has been established for several types of cancers, including squamous cell head and neck cancer (hereinafter referred to as “H&NSCC”), lung, renal-cell, breast, and colorectal cancers (Gabrilovich, 1997; Chaux, 1996; Almand, 2000; Nestle, 1997; Tas, 1993; Thurnher, 1996; Hoffmann, 2002). Characterized dendritic cell defects result in a failure to effectively and successfully present tumor antigens to T cells, and such defects can be characterized in a variety of ways including down-regulation of components of the antigen-processing machinery, reduced expression of co-stimulatory molecules, and a reduction in the number of dendritic cells that infiltrate the tumor (Whiteside, 2004; Gabrilovich, 1997; Choux, 1997). Cancer patients also show a decrease in the absolute numbers of mature DCs in the peripheral blood and lymph nodes (Hoffmann, 2002; Almand, 2000). VEGF, a soluble factor commonly secreted by tumors, has been shown to increase the induction of apoptosis in dendritic cells and negatively correlates with dendritic cell numbers in the tumor tissue and peripheral blood of patients with many different types of cancer, including H&NSCC (Lissoni, 2001; Saito, 1998; Smith, 2000). Overall, a lack of dendritic cell function negatively impacts current immunotherapeutic strategies and correlates with unsuccessful clinical outcomes. Correcting dendritic cell functional defects would increase the number of mature dendritic cells that can then interact with antigens, e.g., tumor antigens, to present such antigens to T cells for the activation of cell-mediated and antibody-mediated immunity in a patient.

For example, sinus histiocytosis (SH) is a lymph node pathology seen in cancer patients that is characterized by the accumulation in lymph nodes of large histiocytes, which are partially mature dendritic cells that have ingested and processed tumor antigens, but are unable to fully mature and present these tumor peptides to naïve T cells. SH is believed to be caused by a defect in dendritic cell processing. Without the proper presentation of antigen to the T cells, these T cells are incapable of stimulating Th1 and Th2 effector cells, which stimulation normally leads to cell-mediated and antibody-mediated immunity, respectively, in the body.

It would be advantageous to detect the above-described immunologic defects in patients in order to provide effective treatment and determine response to treatment. However, immunologic tests in patients with cancer have had limited usefulness in predicting treatment outcome. Many types of immunologic studies have helped to delineate immunologic defects in patients with cancer on an experimental basis, but few tests have been feasibly applied clinically to diagnose and monitor these patients. Two tests have proved useful: 1) lymphocyte counts, specifically T cells and subsets; and 2) skin reactivity to dinitrochlorobenzene (DNCB) as a test of CMI. The latter test is cumbersome and requires immunization and challenge days later after the skin test and is no longer used clinically. The former is used but not emphasized as a predictor of outcome. Several other DTH skin tests have been developed to diagnose immune deficiency and are further detailed below.

There are two different limbs of the immune system that elicit a DTH or CMI skin test response: 1) the afferent (input) limb; and 2) the efferent (output) limb. The afferent limb involves antigen or mitogen-triggered T cell proliferation and cytokine production. The efferent limb involves cytokine-induced monocyte influx, and monokine production leading to inflammation measured by erythema and induration.

During the 1970's, several groups developed a skin test with the T cell mitogen, phytohemagglutin (PHA). The PHA skin test appeared to provide the same type of information as the DNCB skin test, i.e., responsive patients did well clinically and unresponsive patients did poorly. However, PHA stimulates both limbs of the response, and therefore, a negative PHA skin test can reflect several defects: insufficient T cells, depressed function of T cells, or defect in monocyte function.

Johnston-Early, et al. (1983) teaches a DTH skin test administered to small cell lung cancer patients with five antigens. Skin test reactive patients survived significantly longer than anergic patients, indicating that skin test reactivity was of prognostic utility primarily in otherwise good prognosis patients, whereas anergy was associated with shortened survival. Skin test reactivity/anergy of patients in categories of poor prognosis or intermediate prognosis had no influence on survival.

Birx, et al. (1993) teaches an algorithm for the selection of DTH skin test antigens. The testing scheme was applied to HIV patients and a correlation was found between skin test reactivity and CD4 cell count. Anergy was found to be independently predictive of the development of late-stage disease, AIDS, or death. The algorithm lead to the modification of skin testing protocol by defining the minimum number of antigens required.

U.S. Pat. No. 6,406,699 to Wood (hereinafter the '699 patent) discloses a cancer immunotherapy involving vaccinating a patient with his own cancer cells and an immunologic adjuvant, removing the cancer antigen-primed peripheral blood mononuclear cells (PBL) from the patient, stimulating primed T cells to differentiate into effector lymphocytes in vitro, and infusing the effector cells back into the patient. The focus of the data in the '699 patent was to breast cancer. The '699 patent notes the historical use of DTH skin testing as an assay for cell-mediated immunity and discloses that patients were skin tested after vaccination via a DTH test to determine that the T cells were in fact being primed to the cancer antigen. The theory behind the DTH skin test is that a DTH reaction occurs because some primed cancer antigen-specific T lymphocytes leave the peripheral blood, enter the skin and interact with the cancer antigen and antigen presenting cells to produce a local immune response. The DTH response provides a well-established measure of cell-mediated immunity that has been extensively studied in experiments in animals and humans.

Several tests have also been developed that relate to genome or nucleic acid-based technology for developing personalized medical interventions in subpopulations of patients having particular biological markers. Systems have also been developed for diagnostic assays using DNA microarrays. Several diagnostic tests detect either specific protein markers on the surface of cancer cells or specific protein activity (e.g. increased activity) associated with cancer in patients. These tests, described below, are not related to antigen skin testing as a diagnostic for diagnosing cancer or cellular immune deficiency, or as a predictor for predicting treatment outcome as disclosed by the present invention.

U.S. Pat. No. 6,949,338 to Rheins, et al. and U.S. Patent Application Publication No. 2005/0221334 A1 to Benson relate to a technology wherein skin samples are analyzed for skin diseases. Epidermal samples are obtained by tape stripping and the sample is analyzed for nucleic acid expression that may be predictive of a skin disease, such as psoriasis or dermatitis. This test is focused on nucleic acid or gene expression rather than antigens.

U.S. Patent Application Publication No. 2004/0214233 A1 to Lubman, et al. discloses a protein microarray system that can be used for detecting markers (e.g. antigens, antibodies) in patients with diseases such as cancer. The Lubman application notes that the protein microarrays disclosed therein can be used in the diagnosis of cancer. This application corresponds in part to the teachings of Wang, et al. (N Eng. J. Med. 353:12) who demonstrate the use of protein microarrays to develop autoantibody signatures in cancer patients, wherein a panel of 22 phage peptides was used to test the serum of prostate cancer patients and was more effective at discriminating between prostate cancer samples and controls than the standard prostate-specific antigen (PSA) test.

Applicant developed a skin test utilizing a natural cytokine mixture (NCM) in U.S. Pat. No. 6,482,389 (hereinafter, the '389 patent). The NCM (also referred to herein as IRX-2), has been previously shown by applicant in U.S. Pat. No. 5,698,194 to be effective in promoting T cell development and function in aged, immunosuppressed mice. Specifically, the NCM was shown to decrease the proportion of immature T cells and increase the proportion of mature T cells in the thymus. The NCM included IL-1, IL-2, IL-6, IL-8, IFN-γ, and TNF-α, as well as GM-CSF, G-CSF, IL-3, IL4, IL-5, IL-7, and IL-12 in trace amounts or absent altogether. The '389 patent discloses a method and kit for determining candidates for immunotherapy, for monitoring the effect of immunotherapy, and for analyzing cell-mediated immunity function in a patient. The method of the '389 patent includes performing two intracutaneous skin tests and reading the skin test after twenty-four hours. One skin test is the administration of a mitogen such as PHA, concanavalin A (ConA), pokeweed antigen (PWA) and other mitogens as known in the art. Response to the PHA skin test reflect the ability of the present T lymphocytes to react to PHA, to release cytokines such as IL-2, and to induce a monocyte and macrophage infiltration leading to the DTH dermal reaction that is observed in the skin test, characteristic of the afferent limb response of the immune system. The second skin test is the administration of NCM and reflects the ability of preformed T cell cytokines to induce the monocyte and macrophage accumulation characteristic of the efferent limb response. The method of the '389 patent essentially provides a process of monitoring patients with cellular immune deficiency by the steps of determining the result of intracutaneous skin tests with a mitogen such as PHA and with NCM and the result of blood lymphocyte counts (with or without T-lymphocyte and subset enumeration) to yield a composite “three-dimensional view” of cellular mediated immunity including T lymphocyte number and function (afferent limb) and cytokine production and action on monocytes and macrophages (efferent limb).

Previously, the NCM skin test in conjunction with the PHA skin test was used to predict only a poor response to NCM immunotherapy. In U.S. patent application Ser. No. 10/637,869, presently allowed and to which this application claims priority, applicant provided data indicating that cancer patients having a negative intradermal skin test reaction to the NCM of the subject invention predicts not only a poor response to immunotherapy but also a poor overall clinical prognosis. However, a certain number of patients were converted from a negative skin test response to a positive one upon treatment with NCM and these converted patients showed improved clinical and pathological responses. It was suggested that a negative skin test to NCM reflects a monocyte defect in the patient, whereby cell-mediated immune responses were deficient, and treatment with NCM can remedy this functional defect. These experiments are detailed in the examples provided herein.

There remains a great need for tests, specifically, diagnostics and personalized therapeutics based on reaction to specific antigens, that will reflect the cancer patient's cellular immune status. Thus, one of the objectives of the present invention is to provide a skin test that reflects the efferent limb response, i.e., the monocyte-dependent component of the immune response. The '389 patent referenced above discloses a PHA skin test performed in conjunction with an NCM skin test. These tests were only performed together, i.e. the NCM skin test was always performed with a PHA test, never separately. There has been no disclosure of the use of an NCM skin test by itself to predict patient response. An NCM composition is therefore provided for use as a diagnostic skin test for predicting treatment outcome, e.g., in cancer patients. As shown herein, it was previously unknown that other T cell mitogens can be used for predictive skin tests including anti-CD3 monoclonal antibodies. A diagnostic skin test of anti-CD3 monoclonal antibodies is therefore also provided herein.

### SUMMARY OF THE INVENTION

The present invention provides a diagnostic skin test for predicting treatment outcome, essentially including an effective amount of an NCM or a T lymphocyte mitogen of muromonab-CD3.

The present invention further provides kits for performing a skin test, including an effective amount of an NCM or a T lymphocyte mitogen of muromonab-CD3.

The present invention also provides a method of performing a skin test on a patient, including the steps of administering an effective amount of an NCM or a T lymphocyte mitogen of muromonab-CD3 to skin, analyzing results of the skin test, and predicting a treatment outcome.

The present invention provides a method of detecting defects in monocyte function, including the steps of administering an effective amount of an NCM to skin, analyzing results of the skin test, and detecting at least one defect in monocyte function.

The present invention also provides a method of detecting defects in T lymphocyte function, including the steps of administering an effective amount of a T lymphocyte mitogen of muromonab-CD3 to skin, analyzing results of the skin test, and detecting at least one defect in monocyte function.

A mechanism is also provided for indicating either a functioning efferent limb or afferent limb of an immune system including a diagnostic skin test with an effective amount of an NCM or a T lymphocyte mitogen of muromonab-CD3, respectively.

### BRIEF DESCRIPTION OF THE DRAWINGS

Other advantages of the present invention are readily appreciated as the same becomes better understood by reference to the following detailed description when considered in connection with the accompanying drawings wherein:

FIG. 1 is a bar graph showing lymph node size in normal controls, cancer controls or NCM-treated populations with H&NSCC;

FIG. 2A is a bar graph showing T cell area and FIG. 2B shows density in normal controls, H&NSCC controls and H&NSCC patients treated with NCM;

FIG. 3A is a bar graph comparing B cell area and FIG. 3B is a bar graph comparing follicles in the three treatment groups;

FIG. 4A shows a comparison of other cells and FIG. 4B shows a comparison of sinus histiocytosis in the three treatment groups;

FIG. 5 is a graph showing a Node B&T (B cell and T cell) and Tumor B&T fit plot;

FIG. 6 is a line graph comparing disease-specific survival over 24 months of three groups of skin test patients: on protocol patients, skin test-negative off protocol patients, and skin test-positive off protocol patients;

FIG. 7A contains two bar graphs depicting the increase in percentage of monocytes and macrophages staining positive for the combination of activation markers, CD86, HLA-DR, CD80 and CD40, after treatment of adherent PBMCs with NCM, as determined by flow cytometry;

FIG. 7B is a series of bar graphs depicting the increase in mean fluorescence intensity (MFI) for the activation markers, CD86, HLA-DR, CD80 and CD40, after treatment of adherent PBMCs with NCM, as determined by flow cytometry;

FIG. 8 contains bar graphs demonstrating that the NCM of the invention activates monocytes and macrophages, i.e., induces the expression of activation markers, CD86, HLA-DR, CD80 and CD40, to a greater degree than TNF-α;

FIG. 9 contains bar graphs demonstrating that the NCM of the invention activates monocytes and macrophages, i.e., induces the activation markers, HLA-DR, CD86 and CD40, even in the presence of the immunosuppressing cytokine IL-10. The NCM is better at activating monocytes and macrophages than LPS, both in the presence and absence of IL-10;

FIG. 10 is a bar graph demonstrating that the NCM of the invention stimulates the production of TNF-α from activated monocytes and macrophages and overcomes the immunosuppressive effects of IL-10. The NCM stimulated the production of TNF-α to a greater extent than LPS; and

FIG. 11 is a line graph showing disease-specific survival over 48 months for skin test-negative patients.

### DETAILED DESCRIPTION OF THE INVENTION

The present invention provides herein compositions including a natural cytokine mixture (NCM) or a T lymphocyte mitogen of muromonab-CD3 for use as a diagnostic skin test to predict treatment outcome in cancer patients, including response to surgery, overall patient survival, time to recurrence, and time to death. A method is provided by which the NCM compositions of the invention are administered intracutaneously and a response to the NCM is determined, wherein a negative skin test indicates unresponsiveness to NCM and predicts failure of patients to respond to surgery (with or without radiotherapy), overall patient survival, time to recurrence, and time to death. A method is further provided by which the T lymphocyte mitogen of muromonab-CD3 of the invention is administered intracutaneously and a response to the muromonab-CD3 is determined, wherein a negative skin test indicates unresponsiveness to NCM and predicts failure of patients to respond to surgery (with or without radiotherapy), overall patient survival, time to recurrence, and time to death.

A method is also provided of detecting defects in monocyte function by administering an effective amount of an NCM to skin, analyzing results of the skin test, and detecting at least one defect in monocyte function. A method is also provided of detecting defects in T lymphocyte function, by administering an effective amount of a T lymphocyte mitogen of muromonab-CD3 to skin, analyzing results of the skin test, and detecting at least one defect in T lymphocyte function. The present invention includes a mechanism for indicating either a functioning efferent limb or afferent limb of an immune system including a diagnostic skin test with an effective amount of an NCM or a T lymphocyte mitogen of muromonab-CD3, respectively. The compositions and methods according to this embodiment of the invention are useful to determine the appropriate treatment of cancer patients.

As used herein, the term “adjuvant” denotes a composition with the ability to enhance the immune response to a particular antigen. Such ability is manifested by a significant increase in immune-mediated protection. To be effective, an adjuvant must be delivered at or near the site of antigen. Enhancement of immunity is typically manifested by a significant increase (usually greater than ten-fold) in the titer of antibody raised to the antigen. Enhancement of cellular immunity can be measured by a positive skin test, cytotoxic T cell assay, ELISPOT assay for IFN-γ or IL-2, or T cell infiltration into the tumor (as described below).

As used herein, “NCM” denotes a natural cytokine mixture, as defined and set forth in U.S. Pat. Nos. 5,632,983 and 5,698,194. The NCM can include recombinant cytokines. Briefly, NCM is prepared in the continuous presence of a 4-aminoquinolone antibiotic and with the continuous or pulsed presence of a mitogen, which in the preferred embodiment is PHA. More specifically, the NCM of the invention contains six critical components, IL-1, IL-2, IL-6, IL-8, INF-γ, and TNF-α, which act to produce naïve T cells. According to a preferred embodiment of the invention, the NCM contains a concentration of IL-1 that ranges from 60-6,000 pcg/ml, more preferably, from 150-1,200 pcg/ml; a concentration of IL-2 that ranges from 600-60,000 pcg/ml, more preferably, from 3,000-12,000 pcg/ml; a concentration of IL-6 that ranges from 60-6,000 pcg/ml, more preferably, from 300-2,000 pcg/ml; a concentration of IL-8 that ranges from 6,000-600,000 pcg/ml, more preferably, from 20,000-180,000 pcg/ml; and concentrations of IFN-γ and TNF-α that range from 200-20,000 pcg/ml, more preferably, from 1,000-4,000 pcg/ml. Recombinant, natural or pegylated cytokines can be used or the NCM can include a mixture of recombinant, natural or pegylated cytokines. The NCM can further include other recombinant, natural or pegylated cytokines such as IL-12, GM-CSF, and G-CSF. The NCM can be administered during treatment either alone, or in conjunction with cyclophosphamide (CY), indomethacin (INDO), and zinc as detailed below in the examples.

As used herein, the term “response” denotes an answer or result to the skin test. A response is acquired after analysis of the skin test reaction on the patient. Throughout the application, “response” is used synonymously with “result”.

As used herein, the term “skin test” denotes a clinical test performed on a patient which stimulates a response on the patient's skin if a certain set of physiological parameters are present, generally relating to the immune system and a particular disease. The skin tests of the present invention are provided as diagnostic tools and for predicting treatment outcomes.

As used herein, the term “T lymphocyte mitogen” denotes an agent that is capable of stimulating mitosis and lymphocyte transformation. This term is also referred to as a “T cell mitogen”. The T lymphocyte mitogen utilized in the present invention is an anti-CD3 monoclonal antibody muromonab-CD3. Muromonab-CD3 (Ortho Biotech), also known under the trade name ORTHOCLONE OKT3®, is commonly administered to patients receiving organ transplants (such as kidney, heart, or liver transplants) in order to lower the patient's natural immune system. In other words, muromonab-CD3 acts as an immune suppressant. This is necessary to help prevent organ rejection from the body, but it also can make the patient more susceptible to infections. Muromonab-CD3 has not previously been used in the manner of the present invention as a skin test component, as detailed in Example 6 below.

As used herein, the term “tumor associated antigen” denotes a protein or peptide or other molecule capable of inducing an immune response to a tumor. This can include, but is not limited to, PSMA peptides, MAGE peptides (Sahin, 1997; Wang, 1999), Papilloma virus peptides (E6 and E7), MAGE fragments, NY ESO-1 or other similar antigens. Previously, these antigens were not considered to be effective in treating patients based either on their size, i.e., they were considered too small, or they were previously thought to lack immunogenic properties (i.e., they were considered to be self antigens).

The present invention provides an NCM for use in a diagnostic skin test to predict treatment outcome in cancer patients. The NCM administered in the skin test according to the present invention preferably contains the six cytokines of IL-1, IL-2, IL-6, IL-8, IFN-γ, and TNF-α as described above. Recombinant, natural or pegylated cytokines can be used or the NCM can include a mixture of such cytokines. The NCM can further include other recombinant, natural or pegylated cytokines such as IL-12, GM-CSF, and G-CSF. When administered in the skin test, the NCM can be administered at 1-500 units of IL-2 equivalence. Preferably, 0.1 ml of the NCM at a concentration of 4 to 50 units of IL-2 equivalence per ml is administered intradermally.

The NCM skin test of the present invention reflects only the efferent limb response, i.e., the monocyte-dependent component. U.S. patent application Ser. No. 10/637,869 and Example 2 below discuss using the NCM of the invention as a diagnostic skin test for predicting treatment outcome by administering an NCM intracutaneously and determining a response to the NCM within 24 hours. A positive skin test (i.e. erythema) generally indicates that the efferent limb of the immune system is working and positive immune therapy treatment outcome is predicted. A negative skin test generally indicates unresponsiveness to the NCM and immunotherapy and predicts a negative treatment outcome. As the efferent limb relates to monocyte function, a negative response to the skin test predicts at least one defect in monocyte function. The present invention therefore includes a mechanism for indicating a functioning efferent limb of an immune system including the diagnostic NCM skin test. An NCM, by itself, has not been used as an indicator of a functioning efferent limb.

There are several specific treatment outcomes that are predicted through the NCM skin test. For example, the overall survival of the patient can be predicted. This is one novel aspect of the invention. Such a test has never been performed before. A positive response to the NCM skin test favors a major clinical response and that, after treatment with NCM treatment, a patient will survive and remain disease-free. A negative response to the NCM skin test indicates that, even with NCM treatment, a patient's chances of surviving overall are limited. These responses are demonstrated below in Example 3. These predictions are useful in determining personalized therapy. It is helpful to select for good responders to treatment not only for the benefit of the patient to get the proper treatment but also to conserve a limited drug supply. Where there is a limited drug supply, especially, for example, in Third World countries, those who would benefit most from the treatment can be selected first to receive the treatment.

Also, response to immunotherapy is predicted through the NCM skin test. A positive skin test result indicates that a patient will respond to immunotherapy with NCM, whereas a negative skin test result indicates that the patient will not respond to immunotherapy with NCM. Predicting response to immunotherapy is further detailed in Examples 1, 2, and 3 below.

The NCM skin test also predicts response to surgery with or without radiotherapy in combination with NCM treatment, as detailed in Example 3. A positive skin test result predicts that surgery with or without radiotherapy along with NCM treatment will favor a major clinical response and greater survival in a patient. A negative skin test result predicts that surgery with or without radiotherapy along with NCM treatment will not have an impact on a patient's survival.

The NCM skin test further predicts the time to recurrence of disease. A positive skin test result predicts that the time to recurrence will be long, or in other words, recurrence may not occur at all because of the major clinical response predicted. A negative skin test result predicts that the time to recurrence will be short, because the patients will not respond to treatment and their disease will progress. The NCM skin test predicting time to recurrence is further detailed in Example 3 below.

Time to death is also predicted through the use of the NCM skin test. For example, a positive skin test result indicates an elongated period for the time to death of a patient. In other words, the time to death is extended because of the prediction of a positive outcome with NCM treatment. A negative skin test result indicates a shortened period for the time to death of a patient because of predicted non-response to treatment. The NCM skin test predicting time to death is further detailed in Example 3 below.

A negative NCM skin test can be converted into a positive result by pretreatment with NCM, as shown in Example 5. Thus, administration of NCM after a negative skin test result can correct monocyte defects, and a subsequent positive NCM skin test can show an improvement of the immune system and predict a favorable outcome.

The NCM skin test is generally performed by administering an effective amount of NCM to skin, analyzing results of the skin test, and predicting a treatment outcome. The NCM skin test is preferably performed on cancer patients; however, the NCM skin test can also be performed on other immune deficient patients. Administration is generally intradermally, but can be other methods as detailed below. Intradermal injection is preferably into the lower forearm using a 1 cc tuberculin syringe with the needle bevel positioned up. As soon as the bevel is completely covered by intradermal tissue, a small amount of fluid can be injected and the needle advanced slowly administering the remaining volume during advancement. An effective amount of NCM, as stated above, is preferably 4-50 units of IL-2. The results of the skin test are generally analyzed and read from 6 to 48 hours after administration of the test. Preferably, the test results are analyzed and read 24 hours after administration. Either a negative response or a positive response is obtained. A positive response in general predicts a positive treatment outcome. A negative response in general predicts a negative treatment outcome and at least one defect in monocyte function. Thus, the NCM skin test can be administered to detect defects in monocyte function. From the test results, a specific treatment outcome can be predicted as detailed above, including the overall survival of the patient, response to immunotherapy, response to surgery, response to radiotherapy, time to recurrence, and time to death.

According to a second embodiment of the invention, muromonab-CD3 is administered as a diagnostic skin test. Preferably, 0.1 to 100 ng of the muromonab-CD3 is administered during the skin test. The diagnostic skin test with muromonab-CD3 is essentially the same as the NCM skin test described above. A positive response to the muromonab-CD3 skin test generally indicates that the afferent limb of the immune system is functioning as it reflects the ability of the present T-lymphocytes to react to muromonab-CD3, to release cytokines such as IL-2, and to induce a monocyte/macrophage infiltration leading to the DTH dermal reaction which is observed in the skin test. Monocytes and macrophages utilize a common final pathway and aid in antigen presentation for the production of antibodies. Thus a positive response indicates that a positive treatment outcome is predicted. A negative response to the muromonab-CD3 skin test generally indicates unresponsiveness to the NCM and immunotherapy and predicts a negative treatment outcome because of T lymphocyte defects. The present invention therefore includes a mechanism for indicating a functioning afferent limb of an immune system including the diagnostic muromonab-CD3 skin test. While only muromonab-CD3 is described in the present application, it is contemplated that any other T cell mitogen can be utilized in the same manner.

There are several specific treatment outcomes that can be predicted through the muromonab-CD3 skin test. For example, the overall survival of the patient can be predicted. A positive response to the muromonab-CD3 skin test favors a major clinical response and that, after treatment with NCM treatment, a patient will survive and remain disease-free. A negative response to the muromonab-CD3 skin test indicates that, even with NCM treatment, a patient's chances of surviving overall are limited.

Also, response to immunotherapy is predicted through the muromonab-CD3 skin test. A positive skin test result indicates that a patient will respond to immunotherapy with NCM, whereas a negative skin test result indicates that the patient will not respond to immunotherapy with NCM.

The muromonab-CD3 skin test also used to predict response to surgery with or without radiotherapy in combination with NCM treatment. A positive skin test result predicts that surgery with or without radiotherapy along with NCM treatment will favor a major clinical response and greater survival in a patient. A negative skin test result predicts that surgery with or without radiotherapy along with NCM treatment will not have an impact on a patient's survival.

The muromonab-CD3 skin test further predicts the time to recurrence of disease. A positive skin test result predicts that the time to recurrence will be long, or in other words, recurrence may not occur at all because of the major clinical response predicted. A negative skin test result predicts that the time to recurrence will be short, because the patients will not respond to treatment and their disease will progress.

Time to death is also predicted through the use of the muromonab-CD3 skin test. For example, a positive skin test result indicates an elongated period for the time to death of a patient. In other words, the time to death is extended because of the prediction of a positive outcome with NCM treatment. A negative skin test result indicates a shortened period for the time to death of a patient because of predicted non-response to treatment.

A negative muromonab-CD3 skin test can be converted into a positive result by pretreatment with NCM. Thus, administration of NCM after a negative skin test result can correct T lymphocyte defects, and a subsequent positive muromonab-CD3 skin test can show an improvement of the immune system and predict a favorable outcome.

A method of detecting defects in T lymphocyte function, including the steps of administering an effective amount of a T lymphocyte mitogen or muromonab-CD3 to skin, analyzing results of the skin test, and detecting at least one defect in monocyte function.

The muromonab-CD3 skin test is generally performed by administering an effective amount of muromonab-CD3 to skin, analyzing results of the skin test, and predicting a treatment outcome. The muromonab-CD3 skin test is preferably performed on cancer patients; however, the muromonab-CD3 skin test can also be performed on other immune deficient patients. Administration is generally intradermally, but can be other methods as detailed below. Intradermal injection is preferably into the lower forearm using a 1 cc tuberculin syringe with the needle bevel positioned up. As soon as the bevel is completely covered by intradermal tissue, a small amount of fluid can be injected and the needle advanced slowly administering the remaining volume during advancement. An effective amount of muromonab-CD3, as stated above, is preferably 0.1 to 100 ng. The results of the skin test are generally analyzed and read from 6 to 48 hours after administration of the test. Preferably, the test results are analyzed and read 24 hours after administration. Either a negative response or a positive response is obtained. A positive response in general predicts a positive treatment outcome. A negative response in general predicts a negative treatment outcome and at least one defect in T lymphocyte function. From the test results, a specific treatment outcome can be predicted as detailed above, including the overall survival of the patient, response to immunotherapy, response to surgery, response to radiotherapy, time to recurrence, and time to death.

The present invention further provides kits for performing the skin tests described above. A kit for performing the NCM skin test generally includes an effective amount of NCM as described above, preferably 4-50 units of IL-2. The NCM can include cytokines IL-1, IL-2, IL-6, IL-8, IFN-γ, and TNF-α. The NCM can further include cytokines IL-12, GM-CSF, and G-CSF. Further, the cytokines can be recombinant, natural, or pegylated cytokines. The NCM is provided in a pharmaceutically acceptable carrier. The kit also includes the appropriate materials necessary to administer the skin test, such as syringes and needles, as well as control solutions that do not contain the NCM for comparison. The kit can be used to predict any of the treatment outcomes described above of the overall survival of the patient, response to immunotherapy, response to surgery, response to radiotherapy, time to recurrence, and time to death.

A kit for performing the muromonab-CD3 skin test generally includes an effective amount of muromonab-CD3 as described above, preferably 0.1 to 100 ng. The muromonab-CD3 is provided in a pharmaceutically acceptable carrier. The kit also includes the appropriate materials necessary to administer the skin test, such as syringes and needles, as well as control solutions that do not contain the muromonab-CD3 for comparison. The kit can be used to predict any of the treatment outcomes described above of the overall survival of the patient, response to immunotherapy, response to surgery, response to radiotherapy, time to recurrence, and time to death.

For any of the above embodiments, the following administration details and/or protocols for treatment are used:

Preferably, the NCM of the present invention is injected around lymphatics that drain into lymph nodes regional to a lesion, such as a tumor or other persistent lesions being treated. Perilymphatic administration into the lymphatics which drain into the lymph nodes, regional to the lesion, such as a cancer, is critical. Peritumoral injection has been associated with little response, even progression and is thus contraindicated. A ten (10) day injection scheme is optimal and a twenty (20) day injection protocol, while effective clinically, tends to reduce the Th1 response and shift towards a less desirable Th2 response as measured by lymphoid infiltration into the cancer. Bilateral injections are effective. Where radical neck dissection has occurred, contralateral injection is effective.

The compounds of the invention can be administered prior to or after surgery, radiotherapy, chemotherapy, or combinations thereof. The compounds of the invention can be administered during the recurrence of tumors, i.e., during a period where tumor growth is occurring again after a period where tumors were thought to have disappeared or were in remission.

The compounds of the present invention (including NCM) are administered and dosed to promote optimal immunization either to exogenous or endogenous antigen, taking into account the clinical condition of the individual patient, the site and method of administration, scheduling of administration, patient age, sex, and body weight. The pharmaceutically “effective amount” for purposes herein is thus determined by such considerations as are known in the art. The amount must be effective to promote immunization, leading to, e.g., tumor reduction, tumor fragmentation and leukocyte infiltration, delayed recurrence or improved survival rate, or improvement or elimination of symptoms.

In the methods of the present invention, the compounds of the present invention can be administered in various ways. It should be noted that they can be administered as the compound or as a pharmaceutically acceptable derivative and can be administered alone or as an active ingredient in combination with pharmaceutically acceptable carriers, diluents, adjuvants and vehicles. The compounds can be administered intra- or subcutaneously, or per- or intralymphatically, intranodally or intrasplenically or intramuscularly, intraperitoneally, and intrathorasically. Implants of the compounds can also be useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

The doses can be single doses or multiple doses over a period of several days. When administering the compound of the present invention, it is generally formulated in a unit dosage injectable form (e.g., solution, suspension, or emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, can also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it is desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with several of the other ingredients, as desired.

A pharmacological formulation of the present invention can be administered to the patient in an injectable formulation containing any compatible carrier, such as various vehicles, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include those disclosed in: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

The above discussion provides a factual basis for the use of the present invention. The compositions and methods of the invention for use in the utilities disclosed herein can be shown by the following non-limiting examples and accompanying figures.

### EXAMPLES

All steps relating to cell culture are performed under sterile conditions. General methods of cellular immunology not described herein are performed as described in general references for cellular immunology techniques such as Mishell and Shiigi (Selected Methods in Cellular Immunology, 1981) and are well known to those of skill in the art.

Preparation of Natural Cytokine Mixture (NCM)

NCM (also referred to herein as IRX-2) is a defined mixture of cytokines produced under GMP conditions over a 24 hour period following stimulation of human peripheral blood mononuclear cells (PBMCs) by phytohemagglutinin (PHA) and ciprofoxacin. The source of the PBMCs is screened and tested buffy coats purchased from FDA licensed blood banks. After PHA stimulation, the mitogen is removed through centrifugation and washing. All cellular elements are removed by centrifugation, and DNA is removed by anion exchange chromatography. The cell-free supernatant is filter sterilized and nanofiltered to permit viral removal and is designated IRX-2. Stringent QC testing that includes both bioassay and ELISA determination of cytokine levels assures the consistency of the IRX-2. Safety testing with respect to sterility, DNA, mycoplasma, endotoxin and virus testing for CMV and EBV are also part of the GMP process. IRX-2 has been given safely to over 150 patients in various clinical trials and is currently in Phase I/II testing under an FDA approved IND.

More specifically, the NCM can be prepared as follows:

The buffy coat white cells of human blood from multiple HIV-negative hepatitis virus-negative donors are collected. In an alternative embodiment, animals could be the cell source for veterinary uses. The cells from the donors are pooled and layered on ficoll hypaque gradients (Pharmacia) to yield lymphocytes free of neutrophils and erythrocytes. Alternative methods could be used that would result in the same starting lymphocyte population as are known in the art.

The lymphocytes are washed and distributed in X-VIVO 10 media (Whittaker Bioproducts) in surface-activated cell culture flasks for selection of cell subsets. The flasks (MICROCELLECTOR™ T-25 Cell Culture Flasks) contain immobilized stimulants, i.e., mitogens, such as PHA. The immobilization process for the stimulants is as described by the manufacturer for immobilizing various substances for panning procedures, i.e., separating cells, in the flasks. Alternatively, the lymphocytes are exposed to stimulants, e.g., PHA, for 2-4 hours and then washed three times.

The cells are incubated for 24-48 hours in X VIVO-10 media with 80 μg/ml ciprofloxacin (Miles Lab) at 37° C. in a CO₂/air incubator. Alternatively, RPMI 1640 media could be used (Webb et al. 1973). HSA (human serum albumin) may be added to stabilize further the interleukins if HSA-free media is used for generations. Generally, HSA is used at 0.1 to 0.5% (weight by volume). Following incubation the supernatants are poured off and collected. The supernatants are stored at 4° C. to −70° C.

### Example 1

Local perilymphatic injections in the neck with NCM in addition to treatment with low dose CY (at 300 mg/m²), INDO (25 mg orally three times daily), and zinc (65 mg elemental zinc as the sulfate orally once a day) have induced clinical regressions in a high percentage of patients with squamous cell head and neck cancer (H&NSCC) (Hadden, 1994; Meneses, 1998; Barrera, 2000; Hadden, 2003; Menesis, 2003) with evidence of improved, recurrence-free survival. Overall, including minor responses (25%-50%), tumor shrinkage and reduction of tumor in pathological specimens, over 90% responded and the majority had greater than 50% tumor reduction.

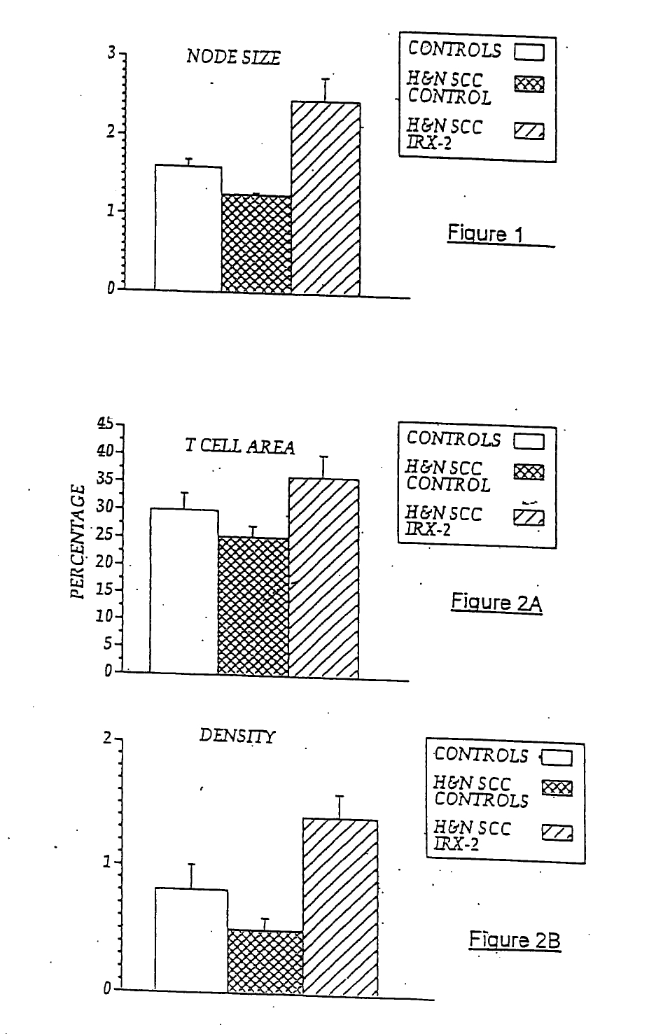

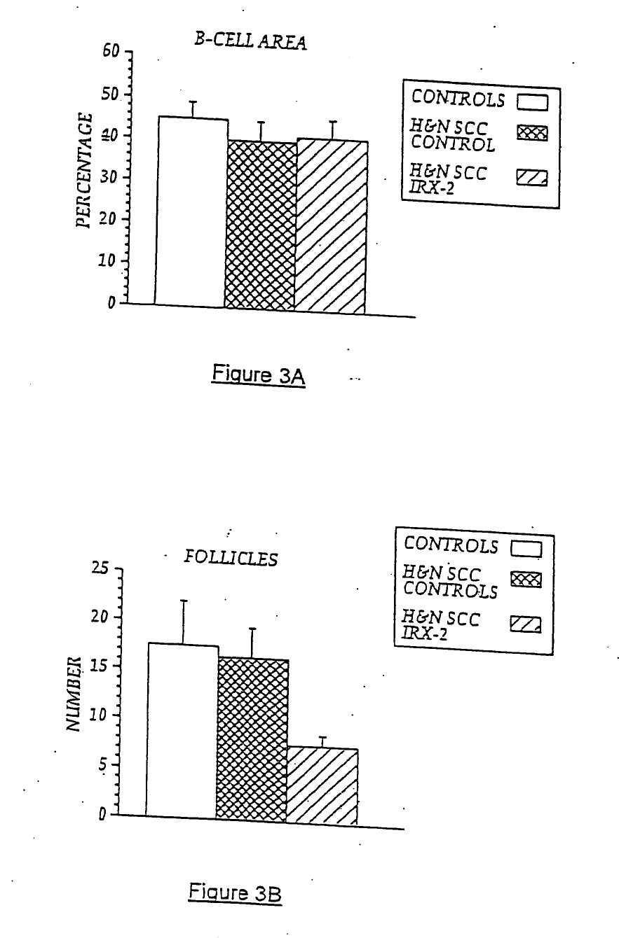

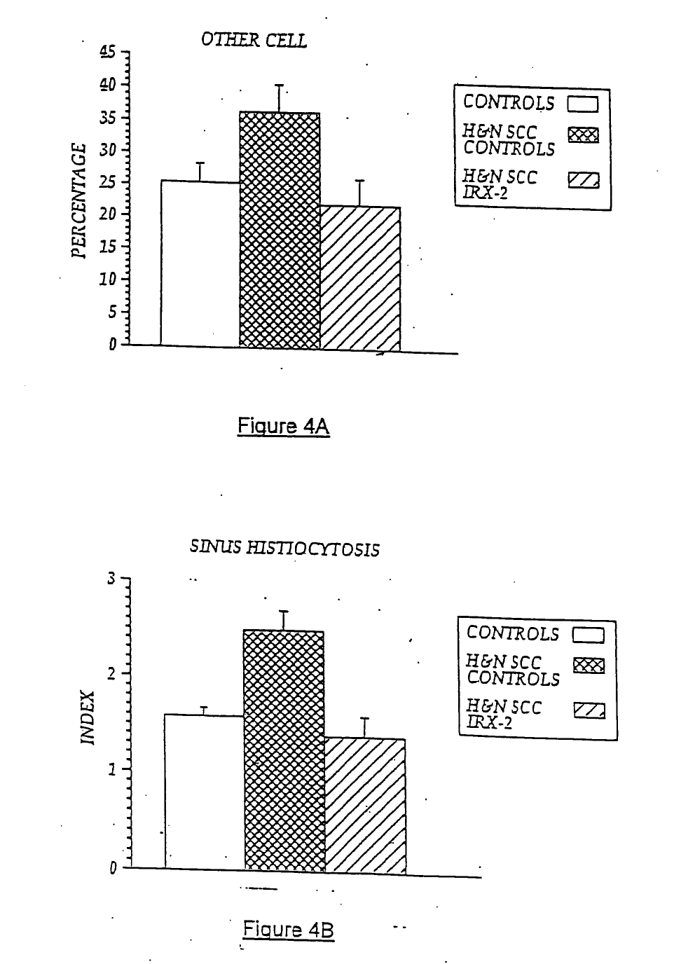

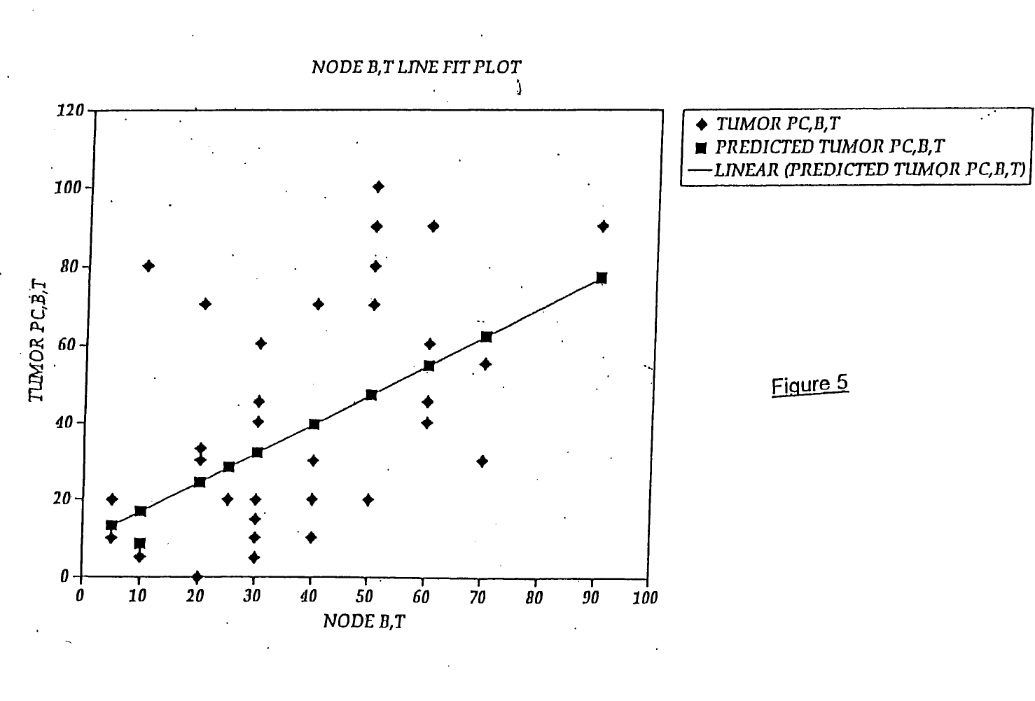

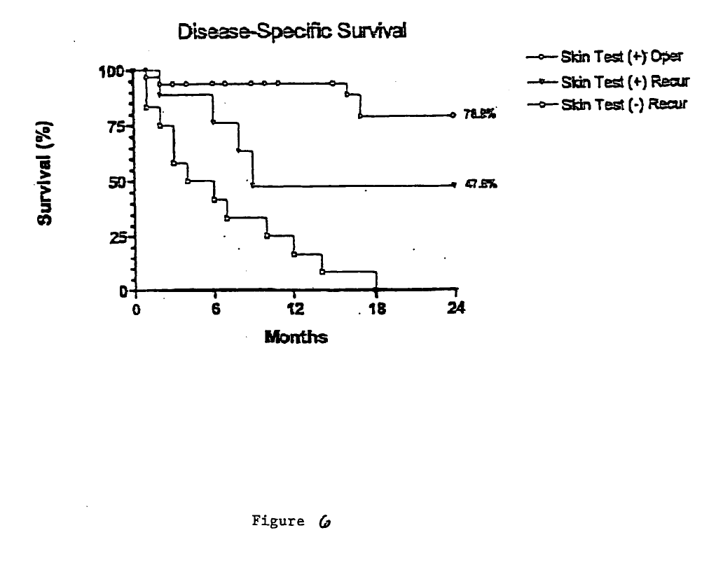

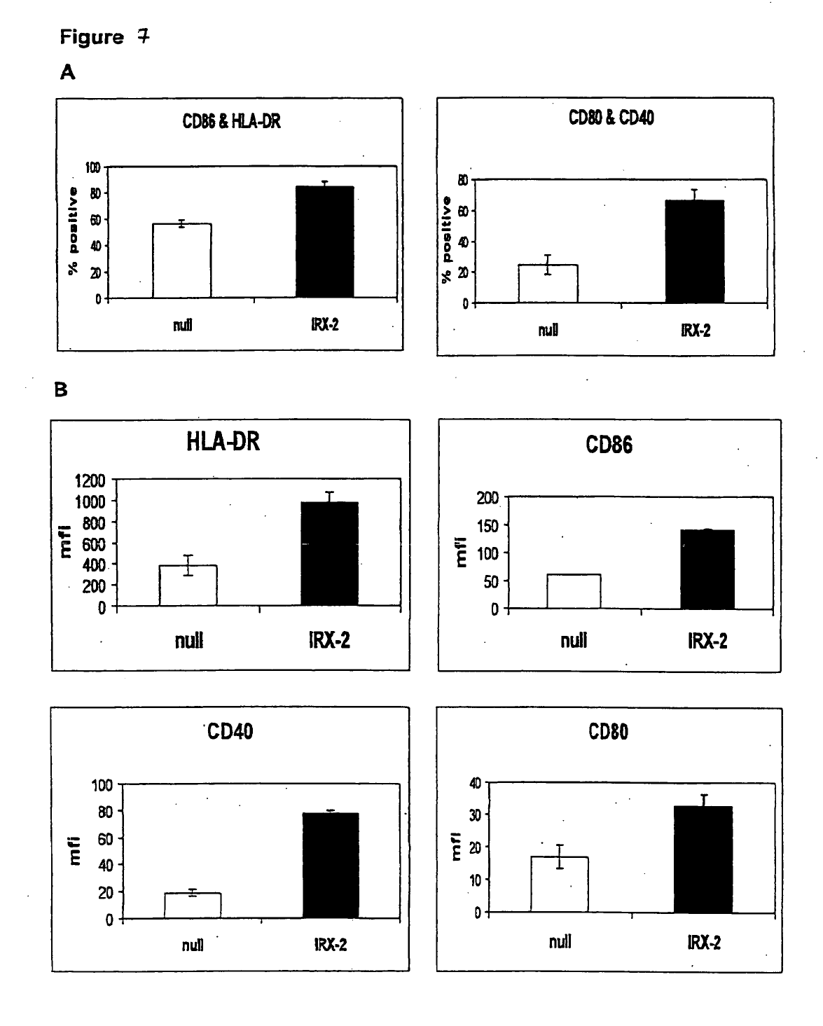

These responses are speculated to be mediated by immune regression since both B and T lymphocytes were observed infiltrating the tumors. The therapy was not associated with significant toxicity. Treatment of lymphocytopenic cancer patients with the combination of NCM has resulted in marked lymphocyte mobilization; where analyzed, these patients showed increases in CD45RA positive T cells (i.e., naïve T cells (see Table I below)). Further, intratumoral or peritumoral injection of NCM in patients with H&NSCC resulted in either reversing immunotherapy-induced tumor regression or in progression of the tumor. The tumor is thus not the site of immunization. Rather, analysis of regional lymph nodes revealed that the regional lymph node is the site of immunization to postulated tumor antigens (Meneses, 2003; see FIGS. 1-5). None of these patients treated with NCM developed distant metastases which would have been expected in 15% of the patients clinically and up to 50% pathologically. These results indicate systemic immunity rather than merely local immunity had been induced. Patients were pretested with a skin test to 0.1 ml of NCM prior to treatment and more than 90% of those with a positive skin test (>0.3 mm at 24 hours) had robust clinical and pathological responses. Patients with negative skin tests had weak or no responses. Thus, skin testing selects good responders.

Major increases were observed in T lymphocyte counts (CD3) 752→1020 in these T lymphocytopoenic patients (T cell counts 752 vs. 1600 (normal)). Importantly, there was a corresponding increase in “naïve” CD45RA positive T cells (532→782). As previously mentioned, these increases are generally not thought to occur in adults particularly with a pharmacological therapy like NCM. These cells presumably are recent thymic emigres and could be considered a major new capacity for responding to new antigens like tumor antigens. The preexisting CD45RA positive cells were not responding to the tumor antigens and may have been incapable of doing so due to tumor-induced immune suppression (anergy).

TABLE I
Treatment of Lymphocytopoenic Patients with H&NSCC with NCM
Increases in Naïve T Cells in Blood (#/mm)

| | NAÏVE T CELL MARKER | | | PAN T CELL MARKER | | |
|---|---|---|---|---|---|---|
| PATIENT # | PRE | POST | INCREASE | PRE | POST | INCREASE |
| 1 | 479 | 778 | +299 | 704 | 1171 | +467 |
| 2 | 938 | 1309 | +371 | 1364 | 1249 | −115 |
| 3 | 98 | 139 | +41 | 146 | 178 | +32 |
| 4 | 341 | 438 | +97 | 655 | 590 | −65 |
| 5 | 567 | 652 | +97 | 453 | 643 | +190 |
| 6 | 658 | 1058 | +400 | 1118 | 1714 | +569 |
| 7 | 642 | 1101 | +459 | 822 | 1601 | +779 |
| MEAN | 532 | 782 | +250 | 752 | 1020 | +269 |

The literature (Hadden J W, Int'l J Immunopharmacol 11/12:629-644, 1997; Hadden J W, Int'l J Immunopharmacol 21:79-101, 1999) indicates that for both SCC and adenocarcinomas, the two major types of cancer, regional lymph nodes reflect abnormalities related to the tumor, including sinus histiocytosis, lymphoid depletion and often the presence of tumor-associated lymphocytes capable of reacting to tumor cells (with IL-2). With metastasis, lymphoid depletion and depressed function occur. A published analysis (Meneses, 2003) of uninvolved cervical lymph nodes in 10 H&NSCC patients and 10 normal controls showed reduction in average lymph node size and an increase in sinus histiocytosis associated with H&NSCC (see FIGS. 1-4A and B of the present application).

Following treatment with one cycle of the NCM protocol (Hadden, 1994; Meneses, 1998; Barrera, 2000), the uninvolved cervical lymph nodes showed the changes indicated in FIGS. 1-4. Compared to the regional lymph nodes of patients with H&NSCC not treated with NCM, these nodes showed a significant increase in size, T cell area and density, and decreases in number of germinal centers, sinus histiocytosis and congestion. The lymph nodes of treated patients were all stimulated and were larger than control nodes with increased T cell area and density. These nodes were thus not only restored to normal but showed evidence of T cell predominance, a known positive correlate with survival in H&NSCC (Hadden, 1997).

Importantly, when the lymph node changes related to B and T cell areas were correlated with the changes in their tumors reflecting T and B cell infiltration, a high degree of correlation was obtained for T cells (p.<0.01) and B cells (<0.01) and overall lymphoid presence (p.<0.001)(FIG. 5). In turn, these changes correlated with tumor reduction by pathological and clinical criteria. These findings indicate that the tumor reactions are directly and positively correlated with lymph node changes and that the tumor reaction reflects the lymph node changes as the dependent variable. These findings, taken in conjunction with knowledge about how the immune system works in general (Roitt I, 1989), and following tumor transfection with a cytokine gene (Maass G, 1995), indicate that the NCM protocol immunizes these patients to yet unidentified tumor antigens at the level of the lymph nodes. No one has previously presented evidence for lymph node changes reflecting immunization with autologous tumor antigens. This confirms that the present invention can induce immunization with previously ineffective or poorly effective tumor antigens in an effect to yield regression of distant metastases.

### Example 2

Role of the Intradermal Skin Test in Prognosis:

We previously suggested that patients with a negative intradermal skin test to NCM might show poor clinical responses based upon a single patient (Hadden, 1994). We have now accumulated a series of skin test negative patients and find that they show responses similar to those observed upon treatment with the CY & INDO combination (without significant NCM) as shown in U.S. patent application Ser. No. 11/374,783. Thus, ten patients had negative skin tests with a NCM of the present invention (i.e., were unresponsive to the NCM) and were subsequently treated with the NCM plus CY and INDO as disclosed in Example 1 above. While these patients had a poor overall clinical response, they nevertheless showed clear cut clinical effects of the CY+INDO treatment including significant lymphoid infiltration, unexpected tumor reduction and fragmentation, and 20% survival (see Table II below).

Importantly, these results also confirm that a positive NCM skin test is critical for predicting the emphatic clinical and pathological responses that relate to improved survival in H&NSCC patients. In addition, a negative skin test predicts the failure of patients to respond to surgery with or without radiotherapy. This is a surprising result, as no other tests have been able to make such a prediction. Knowledge of a likely failure of response can help a patient in deciding a course of treatment, i.e. whether to have surgery and/or radiotherapy that are naturally risky procedures when such procedures are not likely to aid in a clinical prognosis. Thus, the NCM skin test can be usefully employed to predict therapeutic outcome in H&NSCC patients. Previously, skin testing with dinitroclorobenzene (DNCB) showed prognostic significance in H&NSCC, but due to the cumbersome procedure requiring sensitization, it is has ceased to be used clinically. In contrast, the NCM skin test offers a convenient twenty-four hour test.

Interestingly, the patients in our study could be broken down into two groups. In one group, Table IIB, the responses were especially poor with no survivors. In the other group, Table IIA, these patients converted from having a negative NCM test result to having a positive NCM skin test following treatment with NCM (plus CY and INDO) and showed clinical and pathological responses and survival similar to on-protocol patients.

One of these patients had a tumor considered inoperable and was shown to convert from a negative test result to a positive one and upon a second treatment with NCM showed a clinical reduction of the tumor, enhanced pathological responses and prolonged survival following surgery (>7 years). Thus, pretreatment of skin test negative patients with NCM can increase response rates. NCM plus thymosin a, can also be predicted to work (see United States Published Application No. 20030124136). Since a negative NCM skin test reflects a monocyte functional defect, treatment with monocyte-activating cytokines in natural or recombinant form would be predicted to be useful singly or in combination thereof. These include, but are not limited to, GM-CSF, G-CSF, IFN-γ, IL-1, IL-6, IL-8, IL-12 and others. See Example 5 infra, for data relating to the use of NCM to correct monocyte cell functional defects associated with a negative NCM skin test.

TABLE II
Negative NCM Skin Test Patients

| | | | | | | | | | |
|---|---|---|---|---|---|---|---|---|---|
| | | | | | | | Absolute | | |
| Patient | Patient | Tumor | Solid | Frag | Stroma | Lymph. | Tumor | Subj. | |
| No. | Initials | % | % | % | % | % | Reduction | Resp. | Status |

A. Negative NCM Skin Test Changed to Positive

| | | | | | | | | | |
|---|---|---|---|---|---|---|---|---|---|
| 13 | ANA | 48 | 15 | 33 | 16 | 36 | 42 | PR | Alive >24 Mos. |
| 15 | ICV | 70 | 63 | 7 | 6 | 24 | 5 | MR | Alive >24 Mos. |
| 22 | JMM | 50 | 10 | 40 | 10 | 40 | 30 | PR | Died without Disease 9 Mos. |
| 27 | MVR | 70 | 28 | 42 | 1.2 | 18 | 10 | PR | Lost to Follow-up |
| | Mean | 60 | 29 | 31 | 11 | 30 | 22 | | |
| | SD | 12 | 24 | 16 | 4 | 10 | 17 | | |

B. Negative NCM Skin Test

| | | | | | | | | | |
|---|---|---|---|---|---|---|---|---|---|
| 29 | JISM | 80 | 80 | 0 | 10 | 10 | 0 | NR | Died of Disease <1 Year |
| 30 | AGM | 80 | 48 | 32 | 10 | 10 | 0 | NR | Died of Disease <1 Year |
| 35 | NGS* | 70 | 70 | 0 | 0 | 30 | 0 | NR | Died of Disease <1 Year |
| 36 | GCS* | 50 | 15 | 35 | 10 | 40 | 40 | NR | Died of Disease <1 Year |
| 37 | MJBV* | 80 | 16 | 64 | 16 | 4 | 0 | NR | Died of Disease <1 Year |
| 39 | FHV* | 70 | 28 | 42 | 25 | 5 | 0 | NR | Died of Disease <1 Year |
| | Mean | 72 | 43 | 29 | 12 | 17 | 7 | | |
| | SD | 12 | 28 | 25 | 8 | 15 | 16 | | |

### Example 3

The NCM skin test not only predicts response to NCM treatment, with or without surgery ± radiotherapy, but also predicts overall survival, time to recurrence, and time to death in cancer patients.

Fifty four patients with H&NSCC were treated with a combination immunotherapy using NCM (IRX-2) in low dose by injection at the base of the skull, preceded by an injection of low dose cyclophosphamide (CY, 300 mg/M²) and accompanied with daily oral indomethacin (25 mg tid) and zinc (as StressTabs®) as described by Hadden, et al., 1994 and 2003. Thirty two on protocol patients with stage II-IV operable H&NSCC were treated with a 21-day treatment prior to surgery and, where indicated, additional radiotherapy was given following surgery. These patients were skin test positive to a 0.1 ml dose of intradermal NCM (IRX-2) (containing 11-20 units of IL-2 equivalence) and, where tested, were also skin test positive to an intradermal 0.1 ml dose of PHA (0.05 μg-0.5 μg). 16 additional patients were off protocol due to negative skin tests with IRX-2 and in 5 cases had recurrent, progressive inoperable disease. Four of these patients converted to a positive skin test with NCM (IRX-2) and are here considered skin test positive patients. An additional six patients were skin test positive for NCM (IRX-2) but were not on protocol because of recurrent inoperable disease. Thus, the groups of patients were:

1. 32 on protocol patients

2. 12 skin test negative off protocol patients

3. 10 skin test positive off protocol patients

These patients were compared for clinical response to the immunotherapy at the time of surgery, if operated, or at the time of maximal response, if treated with multiple cycles of NCM (IRX-2), as well as for survival at 24 months. Clinical responses were considered major if greater than 50% tumor shrinkage occurred and minor or no responses if there was less than 50% tumor shrinkage (MR/NR).

Results:

Of the 32 on protocol patients, 13 or 42% had major responses. Of the 10 off protocol patients with positive NCM (IRX-2) skin tests, 7 (70%) had major responses. Of the 12 off protocol patients with negative NCM (IRX-2) skin tests, 0 (0%) had major responses. The Chi square analysis comparing the latter two groups is significant (p<0.0005). Thus, a negative NCM skin test predicts the lack of a major response to treatment with immunotherapy. A positive skin test favors but does not ensure a major clinical response.

The results of these three groups on survival are presented in FIG. 6. The on protocol skin test group shows 78.97% overall survival at 24 months. This survival is greater than the 50% overall survival of site and stage matched controls from the same institution treated with surgery ± radiotherapy without the NCM (IRX-2) regimen. The skin test positive off protocol patients were intermediate; six of these patients had recurrent disease. The skin test negative patients all died with shorter disease-free survival and mean survival times than the other two groups (p<0.01). Not only does the skin test predict the future outcome of a patient's treatment, but it selects out those who die quickly in the first year of treatment (see FIG. 11). If over the course of one or two years, a patient still has a negative skin test, the chances of survival are reduced. If a negative skin test is experienced only at the beginning of treatment, the chance of survival increases. Therefore, therapy can be tailored to a patient based on when a negative skin test result is obtained. The presence of a negative skin test thus predicts not only a lack of impact of immunotherapy on survival but also a lack of impact of surgery ± radiotherapy (RT) on survival.

Prior efforts to predict the outcome of surgery ± RT have suggested the following as important: size of the original tumor, lymph node involvement, extracapsular spread, distant metastases, nutrition, and immune status (see Hadden, 1995 for review). Yet, no single clinical finding or test has singled out clinical failures as selectively as does the NCM skin test. Clearly, more emphatic treatments are needed for these patients and more specifically, treatment designed to reverse the defect underlying the negative NCM skin test.

Overall, 23 patients were skin tested for PHA. Greater than 2 year survival was observed for 64% of skin test positive (9/13) but only 20% of skin test negative patients (2/10) (Chi square p<0.01). Three patients in this series were negative for the PHA skin test yet positive for NCM and only one survived greater than 2 years.

The PHA skin test, while a little less predictive than the NCM skin test, nevertheless offers an additional measure for estimating prognosis. The response to PHA reflects a stimulation of T lymphocytes to make the cytokines present in the NCM and the action of these cytokines then attract monocytes into the lesion, causing the delayed hypersensitivity dermal reaction (e.g., the tuberculin reaction). PHA is not approved in the U.S. for use as a diagnostic test, not because it is not safe or effective, but because no company has prepared it for clinical use and done the studies required by the U.S. Food and Drug Administration (FDA). Any agent which is mitogenic for T lymphocytes would be expected to produce this type of skin test reaction. A case in point is anti-CD3 monoclonal antibody, which is clinically available as ORTHOCLONE®, further described in Example 6.

### Example 4

Other Uses of the Present Invention for Prognosis:

Historically, there have been few predictors for outcome (positive or negative) in H&NSCC; lymphocyte counts, 1 gE and 1 gA levels or nutrition were suggested and as mentioned, a DNCB skin test has been used. For chemotherapy (5 FU & cisplatinum), clinical responses occur prior to surgery in the majority of patients, yet mean survival time and overall survival are essentially unaffected. The data presented in the present examples shows that use of the invention delays recurrence of metastasis in those who have residual tumor after surgery and increases survival in a way that relates to the magnitude of the clinical response and the intensity of the immune assault on the tumor as assessed by quantitation of tumor reduction, fragmentation and lymphoid infiltration. These observations point to important modifications of the invention to further improve survival.

In Patients with Severe Immunodeficiency

In patients with low lymphocyte counts, weak or absent NCM skin tests, sinus histiocytosis, and/or poor pathological responses, retreatment with NCM and monitoring of immune responses would be indicated.

In Patients with Minor or No Clinical Responses:

These patients have a high risk of recurrence of metastasis and thus would logically benefit from post surgical treatment with the NCM of the present invention. In the absence of currently available tests for tumor rejection response observed in the patients, follow up testing with the triad of tests individually or collectively described in U.S. Pat. No. 6,482,389 would help to determine the frequency of retreatment with the NCM of the present invention.

In Patients with Recurrent Disease:

Significant responses were observed including two complete responses in patients who were re-treated with the NCM of the present invention. This is in contrast to previous results with natural and recombinant IL-2, wherein such patients failed to respond to retreatment. Thus, the present invention is useful for treating recurrence of disease in patients.

### Example 5

Correction of a Monocyte Functional Defect Characterized by a Negative NCM Skin Test

The role of the intradermal skin test in prognosis was outlined in Examples 2 and 3 above. That data indicated that a negative NCM skin test, i.e., lack of a proliferative T cell response, represents a monocyte defect. Applicant showed that treatment with NCM, INDO, and CY reversed this defect in some patients in whom clinical and histopathological responses and survival increased. At that time, applicant did not know which of the above agents was responsible for the reversal of the monocyte defect. Applicant herein presents data showing that NCM containing the six cytokines of IL-1, IL-2, IL-6, IL-8, IFN-γ, and TNF-α is a potent activator of monocytes/macrophages, i.e., when administered by itself (without the administration of CY or INDO).

More specifically, adherent PBMCs were grown overnight in X-VIVO 10 media (BioWhittaker Bioproducts), stimulated for 24 hours with NCM (IRX-2) (at a 1:3 final concentration) and assayed for the expression of various activation markers typically found on activated macrophages by flow cytometry. As a control, cells were incubated for 24 hr in media lacking NCM. As demonstrated in FIGS. 7A and 7B, the treatment of the cells with NCM versus no added cytokines produced a statistical increase in the percentage of cells staining positively (FIG. 7A) and an increase in mean fluorescence index (MFI) (FIG. 7B) for HLA-CR, CD86, CD40 and CD80, all activation markers of monocytes/macrophages (p<0.03). The data shown in FIGS. 7A and 7B represents the mean value ±SEM from three independent experiments/donors.

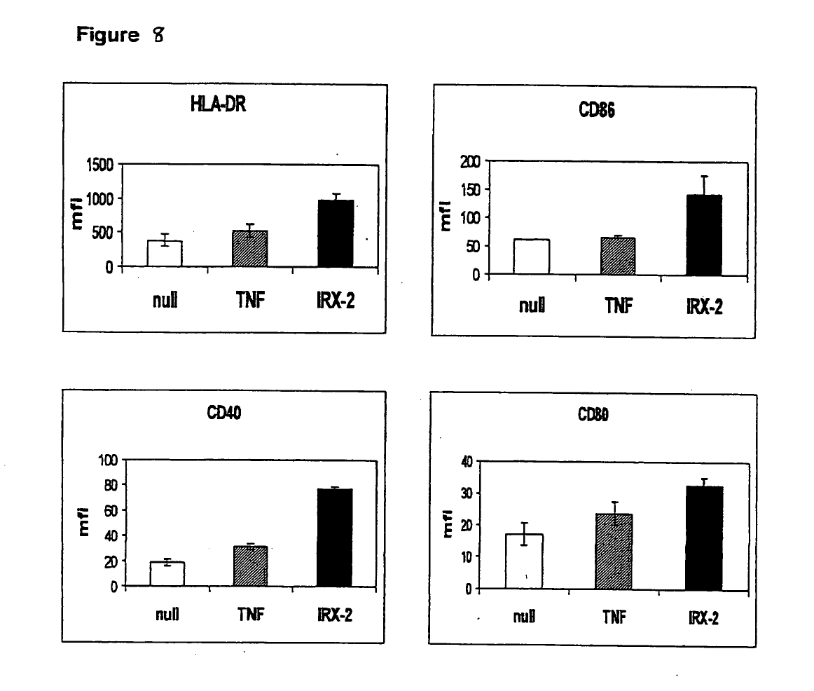

In addition, it was found that the NCM of the invention activates monocytes to a greater degree than TNF-α. More specifically, adherent PBMCs were stimulated with either NCM (IRX-2) (at a 1:3 final concentration; approximately 1 ng/ml TNF-α) or TNF-α (10 ng/ml) and assayed for the expression of activation markers by flow cytometry. As shown in FIG. 8, NCM induced statistically greater expression of HLA-DR, CD86, CD40 and CD80 than TNF-α (p<0.03). The data shown in FIG. 8 represents the mean value ±SEM from three independent experiments/donors.

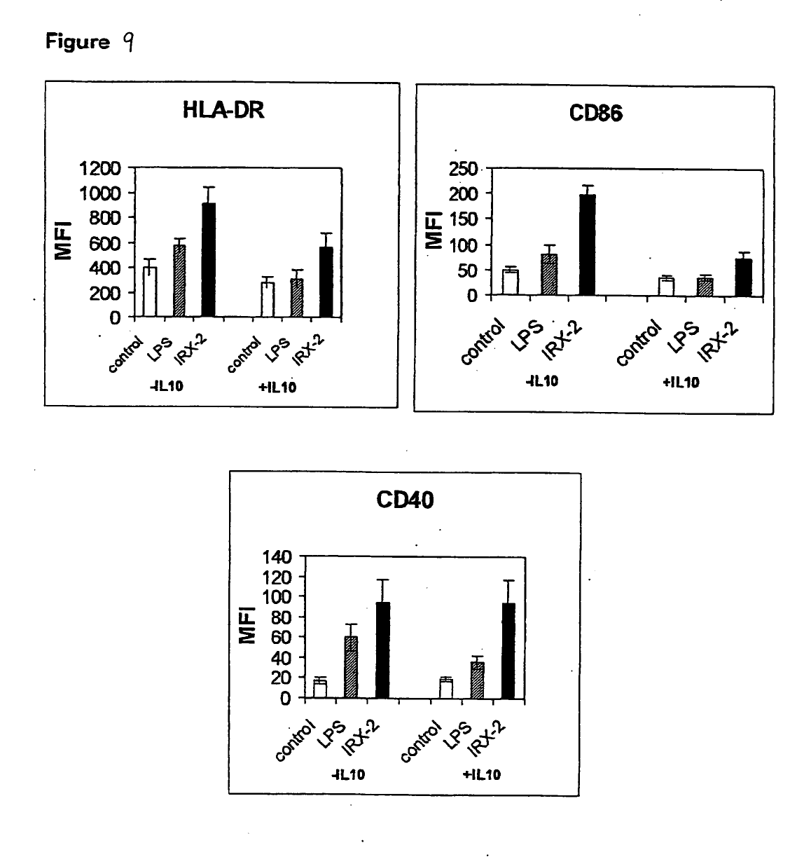

Similarly, studies performed using LPS in modest doses (activating but not maximal) also indicated that NCM was a comparatively stronger activation signal. More specifically, adherent PBMCs were stimulated in the absence or presence of IL-10 (5 ng/ml) with either NCM (IRX-2) (at a 1:3 final concentration) or LPS (10 ng/ml) and assayed for the expression of activation markers by flow cytometry. As shown in FIG. 9, NCM caused a greater increase in the expression of the monocyte/macrophage maturation markers HLA-DR, CD86, and CD40 than LPS. Moreover, in the presence of the immunosuppressing cytokine, IL-10, the NCM was still able to stimulate the monocytes, whereas LPS failed to do so (p<0.02). The data shown in FIG. 9 represents the mean value ±SEM from three independent experiments/donors.

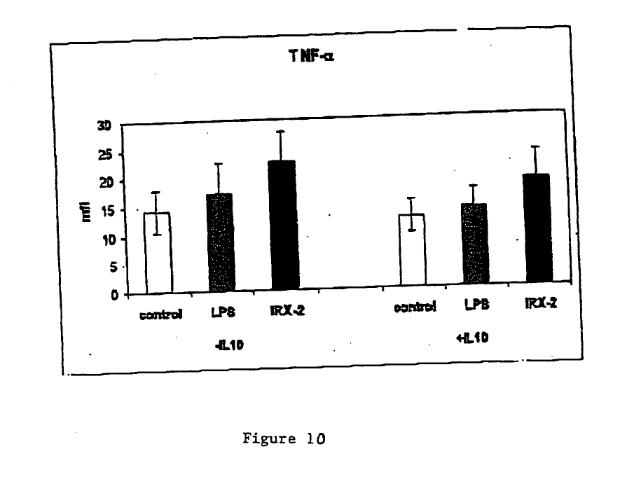

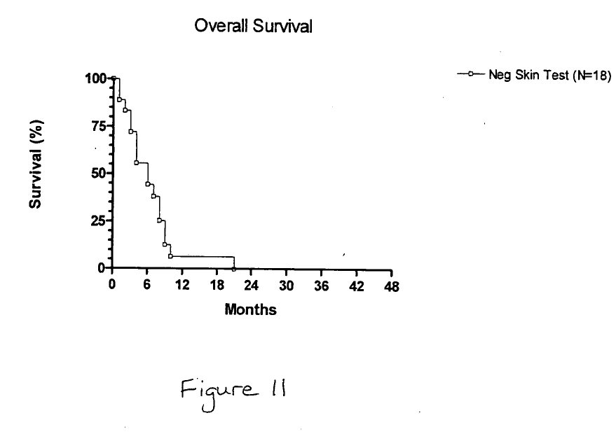

Finally, it is known that monocytes secrete TNF-A in response to activating signals, which secretion is associated with the non-specific killing of tumor cells by the monocytes/macrophages. The data shown in FIG. 10 demonstrates that the NCM of the invention stimulates the production of TNF-α from monocytes and overcomes the immunosuppressive effects of IL-10. More specifically, adherent PBMCs were stimulated in the absence or presence of IL-10 (5 ng/ml) with either NCM (IRX-2) (at a 1:3 final concentration) or LPS (10 ng/ml) and assayed for TNF-α production by intracellular staining and flow cytometry. As shown in FIG. 10, NCM caused a greater increase in the production of TNF-α than LPS or controls. In the presence of IL-10, the NCM was still able to stimulate the monocytes to produce TNF-α, whereas LPS was no longer able to do so (p<0.05). The data shown in FIG. 10 represents the mean value ±SEM from five independent experiments/donors.

These observations of the monocytes' reaction to NCM were unique and have not been previously shown. The reversal of IL-10 effects by the NCM shows the reversal of known tumor-induced defects. Therefore, NCM treatment can reverse a known tumor-induced defect caused by IL-10 producing CD4+ Tregs. The fact that NCM alone has been shown to be a potent activator of monocytes/macrophages supports the contention that NCM treatment alone is responsible for correction of one or more monocyte functional defects characteristic of cancer patients, such as those having a negative NCM skin test.

### Example 6

Muromonab-CD3 (ORTHOCLONE OKT-3®) Skin Test

Three normal human patients were tested with intradermal skin tests of 0.1 ml of anti-CD3 monoclonal antibody ORTHOCLONE OKT-3®, which is known to be a T cell mitogen in culture at low doses and a T cell suppressant (i.e., an immunosuppressive agent) at high doses in vivo. Skin tests were performed with 1, 10, and 100 ng of ORTHOCLONE OKT-3® and read at 24 hours. Positive reactions to one or more of the doses were observed with approximately 1 cm of erythema and induration. This is the first demonstration that a T cell stimulant other than phytohemagglutin (PHA) can cause a positive skin test and reflect T cell response and efferent limb activation. These data predict that all T cell mitogens will have this reaction and will be useful as a new diagnostic test for the cell mediated immune system.

Throughout this application, various publications, including U.S. patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the described invention, the invention can be practiced otherwise than as specifically described.

### REFERENCES

U.S. Pat. Nos.

4,116,951
4,353,821
4,390,623
4,439,196
4,447,224
4,447,233
4,464,355
4,466,918
4,470,926
4,475,196
4,486,194
4,487,603
4,612,365
4,910,296
4,925,678
4,959,217
5,100,664
5,167,616
5,169,383
5,225,182
5,503,841
5,632,983
5,643,565
5,698,194
5,800,810
6,060,068

### PUBLICATIONS

Albert et al, Nature, Vol. 392, pp. 86-89 (1998).
Almand B, Resser J, Lindman B, Nadaf S, Clark J, Kwon E, Carbone D P, and Gabrilovich D. Clinical significance of defective dendritic cell differentiation in cancer. Clinical Cancer Research, 6:1755-1766 (2000).
Banchereau et al, Annual Reviews of Immunology, Vol. 18, pp. 767-811 (2000).
Barrera J, Verastegui E, Meneses A, de la Garza J, Zinser J & Hadden J W. Neoadjuvant immunological treatment with IRX-2 in patients with advanced oral cavity squamous cell carcinoma of the head and neck induces clinical and histological responses. In First World Congress on Head and Neck Oncology. J J Alvarez Vicent, Ed. Monduzzi, Bologna, Italy; 1998; pp 1017-1019.
Barrera J, Verastegui E, Meneses A, Zinser J, de la Garza J, Hadden J W. Combination immunotherapy of squamous cell head and neck cancer: A phase 11 trial. Arch Otolaryngol Head Neck Surg 126:345-351 (2000).
Belidegrun A, Kasid A, Uppenkamp M, Topalian S L, and Rosenberg S A. Human tumor infiltrating lymphocytes: analysis of lymphokine mRNA expression and relevance to cancer immunotherapy. Journal of Immunology, 42:45204526 (1989).
Bellone, et al, Immunology Today, Vol 20, No. 10, p 457-462 (1998).
Bender A, Sapp M, Schuler G, Steinamn R, and Bhardwaj N. Improved methods for the generation of dendritic cells from nonproliferating progenitors in human blood. Journal of Immunological Methods, 196:121-135.
Berd D, Maguire Jr H C, Mastrangelo M J. Potentiation of human cell-mediated and humoral immunity by low-dose cyclophosphamide. Cancer Res 1984; 44:5439-43.
Berd D, Mastrangelo M J. Effect of low dose cyclophosphamide on the immune system of cancer patients: reduction of T suppressor function without depletion of the CD8+subset. Cancer Research 47:3317-3321 (1987).
Berd D. Low doses of chemotherapy to inhibit suppressor T cells. Progress in Clin Biol Res 288:449458 (1989).
Berchtold S, Muhl-Zurbes P, Heufler C, Winklehner P, Schuler G and Steinkasserer. Cloning, recombinant expression and biochemical characterization of the murine CD83 molecule, which is specifically upregulated during dendritic cell maturation. FEBS Letters, 461:211-216 (1999).
Birx D L, Brundage J, Larson K, Engler R, Smith L, Squire E, Carpenter G, Sullivan M, Rhoads J, Oster C, et al. The prognostic utility of delayed-type hypersensitivity skin testing in the evaluation of HIV-infected patients. J Acquir Immune Defic Syndr. 6(11):1248-57 (1993).
Borysiewickz L K, Fiander A. Nimako M. A recombinant vaccine virus encoding human papilomavirus type 16 and 18, E6 and E7 proteins as immunotherapy for cervical cancer. Lancet 347:1524-1527 (1996).
Burke and Olson, “Preparation of Clone Libraries in Yeast Artificial-Chromosome Vectors” in Methods in Enzymology, Vol. 194, “Guide to Yeast ‘Genetics and Molecular Biology”, eds. C. Guthrie and G. Fink, Academic Press, Inc., Chap. 17, pp. 251-270 (1991).
Capecchi, “Altering the genome by homologous recombination” Science 244:1288-1292 (1989).
CelIa M, Scheidegger D, Palmer-Lehamann K, Lane P, Lanzavecchia A, and Alber G. Ligation of CD40 on dendritic cells triggers production of high levels of interleukin-12 and enhances T cell stimulatory capacity: T-T help via APC activation. Journal of Experimental Medicine, 184:747 (1996).
Cella M, Engering A, Pinet V, Pieters J, and Lanzavecchia A. Inflammatory stimuli induce accumulation of MHC II complexes on dendritic cells. Nature, 388:782 (1997).
Chaux P, Moutet M, Faivre J, Martin F, and Martin M. Inflammatory cells infiltrating human colorectal carcinomas express HLA class 11 but not B7-1 and B7-2 costimulatory molecules of the T-cell activation. Laboratory Investigations, 74:975-983 (1996).
Cortesina G, DeStefani A, Galcazzi E. Temporary regression of recurrent squamous cell carcinoma of the head and neck is achieved with a low dose but not a high dose of recombinant interleukin 2 injected perilymphatically. Br J Cancer 69:572-577 (1994).
Cortesina G, DeStefani A, Giovarelli M, et al. Treatment of recurrent squamous cell carcinoma of the head and neck with low doses of interleukin-2 injected perilymphatically. Cancer 62:2482-2485 (1988).
Cortesina G, Destefani A & Galeazzi E. Temporary regression of recurrent squamous cell carcinoma of the head and neck is achieved with a low but not high dose of recombinant interleukin-2 injected perilymphatically. Br. J. Cancer; 1994; 69: 572-577.
Cowens J W, Ozer H, Ehrke M J, Colvin M, Mihich E. Inhibition of the development of suppressor cells in culture by 4-hydroperoxycyclophosphamide. J Immunol 1983; 132:95-100.
Cozzolino F, Torcia M, Carossino A M, Giordani R, Selli C, Talini G, Reali E, Novelli A, Pistole V, and Ferrarini M. Characterization of cells from invaded nodes in patients with solid tumors. Lymphokine requirement for tumor-specific lymphoproliferative response. Journal of Experimental Medicine, 166:303-318 (1987).
Cregg J M, Vedvick T S, Raschke W C: Recent Advances in the Expression of Foreign Genes in Pichia pastoris, Bio/Technology 11:905-910 (1993).
Cross D S, Platt J L, Juhn S K, et al. Administration of a prostaglandin synthesis inhibitor associated with an increased immune cell infiltrate in squamous cell carcinoma of the head and neck. Arch Otolaryngol Head Neck Surg 1992; 118: 526-8
Culver, Site-Directed recombination for repair of mutations in the human ADA gene. (Abstract) Antisense DNA & RNA based therapeutics (1998).
Davies et al., “Targeted alterations in yeast artificial chromosomes for inter-species gene transfer”, Nucleic Acids Research, Vol. 20, No. 11, pp. 2693-2698 (1992).
DeLaugh and Lofts, Current Opinion In Immunology, Vol. 12, pp. 583-588 (2000).
DeStefani A, Forni G, Ragona R, et al. Improved survival with perilymphatic interleukin 2 in patients with resectable squamous cell cancer of the inner cavity and oropharynx. Cancer 2002; 95: 90-97
de Vries I J M, Krooshoop D J E B, Scharenborg N M, Lesterhuis W J, Diepstra J H S, van Muijen G N P, Strijk S P, Ruers T J, Boerman O C, Oyen W J G, Adema G J, Punt C J A, and Figdor C G. Effective Migration of Antigen-pulsed Dendritic Cells to Lymph Nodes in Melanoma Patients Is Determined by Their Maturation State. Cancer Research, 63:12-17 (2003).
Dickinson et al., “High frequency gene targeting using insertional vectors”, Human Molecular Genetics, Vol. 2, No. 8, pp.1299-1302 (1993).
Duff and Lincoln, “Insertion of a pathogenic mutation into a yeast artificial chromosome containing the human APP gene and expression in ES cells”, Research Advances in Alzheimer's Disease and Related Disorders (1995).
Ehrke M J, Immunomodulation in cancer therapies. International Immunopharmacology 2003; 3:1105-19.
Faanes R B, Merluzzi V J, Ralph P, Williams N, Tarnowski G S. Restoration of tumor and drug-induced immune dysfunction. In: Serrou B, Rosenfeld C, editors. International Symposium on New Trends in Human Immunology and Cancer Immunotherapy. Paris: Doin Editeurs; 1980. p. 953-64.
Gabrilovich D, Ciernik F, and Carbone D P. Dendritic cells in anti-tumor immune responses. Defective antigen presentation in tumor-bearing hosts. Cellular Immunology, 170:101-110 (1996a).
Gabrilovich D, Chen H, Girgis K, Cunningham T, Meny G, Nadaf S, Kavanaugh D, and Carbone D P. Production of vascular endothelial growth factor by human tumors inhibits the functional maturation of dendritic cells. Nature Medicine, 2:1096-1103 (1996b).
Gabrilovich D, Corak J, Ciernik I F, Kavanaugh D, and Carbone D P. Decreased antigen presentation by dendritic cells in patients with breast cancer. Clinical Cancer Research, 3:483 (1997).
Gabrilovich D, Ishida T, Oyama T, Ran S, Kravtsov V, Nadaf S, and Carbone D P. Vascular endothelial growth factor inhibits the development of dendritic cells and dramatically affects the differentiation of multiple hematopoietic lineages in vivo. Blood, 92:4150-4156 (1998).
Gallo O, Franchi A, Magnelli L, Sardi I, Vannacci A, Boddi V, Chiarugi V, and Masini E. Cyclooxygenase-2 Pathway Correlates with VEGF Expression in Head and Neck Cancer. Implications for Tumor Angiogenesis and Metastasis. Neoplasia, 3:53-61 (2001).
Gilboa, E, Eglitis, M A, Kantoff, P W, Anderson, W F: Transfer and expression of cloned genes using retroviral vectors. BioTechniques 4(6):504-512 (1986). Gillis et al. (1978)
Hadden J W, Endicott J, Baekey P, Skipper P, Hadden E M. Interleukins and contrasuppression induce immune regression of head and neck cancer. Arch Otolaryngol Head Neck Surg. 120:395403 (1994).
Hadden J W, Saha A R, Sosa M, Hadden E M. Immunotherapy with natural interleukins and/or Thymosin al potently augments T lymphocyte responses of hydrocortisone-treated aged mice. Int'l J Immunopharmacol 17:821-828 (1995).
Hadden J W, Verastegui E, Barrera J L, Kurman M, Meneses A, Zinser J W, de la Garza J, and Hadden E. A trial of IRX-2 in patients with squamous cell carcinomas of the head and neck. International Journal of Immunopharmacology, 3:1073-1081 (2003).
Hadden J W, Verastegui E, Barrera J, Meneses A, and de la Garza J. Lymph Node Histology in Head and Neck Cancer: Impact of IRX-2 Immunotherapy. Abstract #294 presented at 2004 Annual Meeting of the American Head and Neck Society, Combined Otolaryngology Spring Meetings (COSM), Washington D.C. (2004).
Hadden J W. The immunology of head and neck cancer: prospects for immunotherapy. Clinical Immunotherapy, 3:362-385 (1995a).
Hadden J W. Immunology and immunotherapy of breast cancer: An update: Int'l J Immunopharmacol 21:79-101 (1999).
Hadden J W. The immunopharmacology of head and neck cancer: An update. Int'l J Immunopharmacol 11/12:629-644 (1997).
Hadden J W. The treatment of zinc deficiency is an immunotherapy. Int'l J Immunopharmacol 17:696-701 (1995).
Hadden, J., E. Verastegui, J. L. Barrera, M. Kurman, A. Meneses, J. W. Zinser, J. de la Garza, and E. Hadden, “A trial of IRX-2 in patients with squamous cell carcinomas of the head and neck,” International Immunopharmacology 3; 1073-1081 (2003).
Hank A J, Albertini M R, Sondel P M. Monoclonal antibodies, cytokines and fusion proteins in the treatment of malignant disease. Cancer Chemother & Biol Resp Mod 18:210-222 (1999).
Hart D N. Dendritic cells: unique leukocyte population which control the primary immune response. Blood, 90:3245-3287 (1997).
Hengst J C D, Mokyr M B, Dray S. Importance of timing in cyclophosphamide therapy of MOPC-315 tumor-bearing mice. Cancer Res 1980; 40:2135-41.
Hengst J C D, Mokyr M B, Dray S. Cooperation between cyclophosphamide tumoricidal activity and host antitumor immunity in the cure of mice bearing large MOPC-315 tumors. Cancer Res 1982; 41:2163-7.
Hirsch B, Johnson J T, Rabin B D, et al. Immunostimulation of patients with head and neck cancer. Arch Otolaryngol 1983; 109: 298-301
Hoffmann T, Muller-Berghaus J, Ferris R, Johnson J, Storkus W, and Whiteside T. Alterations in the Frequency of Dendritic Cell Subsets in the Peripheral Circulation of Patients with Squamous Cell Carcinomas of the Head and Neck. Clinical Cancer Research, 8:1787-1793 (2002).
Holtl L, Zelle-Rieser C, Gander H, Papesh C, Ramoner R, Bartsch G, Rogatsch H, Barsoum A L, Coggin JH Jr., and Thurnher M. Immunotherapy of Metastatic Renal Cell Carcinoma with Tumor Lysate-pulsed Autologous Dendritic Cells. Clinical Cancer Research, 8:3369-3376 (2002).
Huston et al, “Protein engineering of single-chain Fv analogs and fusion proteins” in Methods in Enzymology (J J Langone, ed.; Academic Press, New York, N.Y.) 203:46-88 (1991).
Huxley et al., “The human HPRT gene on a yeast artificial chromosome is functional when transferred to mouse cells by cell fusion”, Genomics, 9:742-750 (1991).
Jakobovits et al., “Germ-line transmission and expression of a human-derived yeast artificial chromosome”, Nature, Vol. 362, pp. 255-261 (1993).
Johnson and Bird, 1991 “Construction of single-chain Fvb derivatives of monoclonal antibodies and their production in Escherichia coli in Methods in Enzymology (J J Langone, ed.; Academic Press, New York, N.Y.) 203:88-99 (1989).
Johnston-Early A., et al. Delayed hypersensitivity skin testing as a prognostic indicator in patients with small cell lung cancer. Cancer, 52(8):1395400 (1983).
Kavanaugh D Y, Carbone D P. Immunologic dysfunction in cancer. Hematol-Oncol Clinics of North Amer 10(4):927-951 (1996).
Kalinski P, Schuitemaker J, de Jong E, and Kapsenberg M. Prostaglandin E(2) is a selective inducer of interleukin-12 p40 (IL-12p40) production and an inhibitor of bioactive IL-12p70 heterodimer. Blood, 97:3466-3469 (2001).
Kaya M, Wada T, Akatsuka T, Kawaguchi S, Nagoya S, Shindoh M, Higashino F, Mezawa F, Okada F, and Ishii S. Vascular endothelial growth factor expression in untreated osteocarcoma is predictive of pulmonary metastasis and poor prognosis. Clinical Cancer Research, 6:572-578 (2000).
Kleindienst P and Brocker T. Endogenous dendritic cells are required for amplification of T cell responses induced by dendritic cell vaccines in vivo. Journal of Immunology, 170:2817-2823 (2003).
Lamb et al., “Introduction and expression of the 400 kilobase precursor amyloid protein gene in transgenic mice”, Nature Genetics, Vol. 5, pp. 22-29 (1993).
Langenkamp A, Messi M, Lanzavecchia A, and Sallusto F. Kinetics of dendritic cell activation: impact on priming of TH1, TH2 and nonpolarized T cells. Nature Immunology, 1:311-316 (2000).
Lissoni P, Malugani F, Bonfanti A, Bucovec R, Secondino S, Brivio F, Ferrari-Bravo A, Ferrante R, Vigoe L, Rovelli F, Mandal M, Viviani S, Fumagalli L, and Gardani G. Abnormally enhanced blood concentrations of vascular endothelial growth factor (VEGF) in metastatic cancer patients and their relation to circulating dendritic cells, IL-12 and endothelin-1. Journal of Biological Regulatory and Homeostatic Agents, 15:140-144 (2001).
Lou Y, Wang G, Lizee G, Kim G J, Finkelstein S E, Feng C, Restifo N P, Hwu P. Dendritic cells strongly boost the antitumor activity of adoptively transferred T cells in vivo. Cancer Research, 64:6783-6790 (2004).
Maass G, Schmidt W, Berger M, et al. Priming of tumor-specific T-cells in the draining lymph nodes after immunization with interleukin 2-secreting tumor cells: three consecutive stages may be required for successful tumor vaccination. Proc Natl Acad Sci USA, 92:5540-5542 (1995).
Mackall. Stem Cells 2000, Vol.18. pp. 10-18.
Mackall, et al. New England Journal of Medicine, Vol. 332, pp. 143-149 (1995).
Maclean G D, Miles D W, Rubens R D, Reddish M A, Longenecker bone marrow. Enhancing the effect of Theratope STn-KLH cancer vaccine in patients with metastatic breast cancer by pretreatment with low-dose intravenous cyclophosphamide. J Immunother Emphasis Tumor Immunol 19(4):309-316 (1996).
Marshak et al, “Strategies for Protein Purification and Characterization. A laboratory course manual.” CSHL Press (1996).
Mastrangelo M J, Maguire H C Jr., Sato T, Nathan F E, Berd D. Active specific immunization in the treatment of patients with melanoma. (Review) Seminars in Oncology 23(6):773-781 (1996).
Matzinger P. Tolerance, danger, and the extended family. Annual Review of Immunology, 12:991-1045 (1994).
Meneses A, Verastegui E, Barrera J L, Zinser J, de la Garza J, Hadden J W. Histological findings in patients with head and neck squamous cell carcinoma receiving perilympatic natural cytokine mixture prior to surgery. Arch Pathol Lab Med 122:447454 (1998).
Meneses A, Verastegui E, Barrera J L, de la Garza J, and Hadden J W, “Lymph node histology in head and neck cancer: impact of immunotherapy with IRX-2,“International Immunopharmacology, 3; 1083-1091 (2003).
Mernaugh and Mernaugh, “An overview of phage-displayed recombinant antibodies” in Molecular Methods In Plant Pathology (R P Singh and U S Singh, eds.; CRC Press Inc., Boca Raton, Fla.) pp. 359-365 (1995).
Middel P, Fayyazi A, Kaboth U, and Radzun H J. Sinus histiocytosis with massive lymphadenopathy: evidence for its relationship to macrophages and for a cytokine-related disorder. Histopathology, 35:525-533 (1999).
Mishell and Shiigi. Selected Methods in Cellular Immunology (1981).
Miyake M, Taki T, Hitomi S, and Hakomori S. Correlation of expression of H/Le(y)/Le(b) antigens with survival in patients with carcinoma of the lung. New England Journal of Medicine, 327:14-19 (1992).
Mokyr M B, Hengst J C D, Dray S. The role of antitumor immunity in cyclophosphamide-induced rejection of subcutaneous non-palpable MOPC-315 tumors. Cancer Res 1982; 42:974-9.
Montovani A, Sozzani S, Locati M, et al. Macrophage polarization: tumor associated macrophages as a paradigm for polarized M2 mononuclear phagocytes. Trends in Immunol 2002; 23: 549-555
Murphy G P, Tjoa B A, Simmons S J. The prostate. 38:43-78 (1999).
Nestle F, Burg G, Fah J, Wrone-Smith T, and Nickoloff B. Human sunlight-induced basal-cell-carcinoma-associated dendritic cells are deficient in T cell co-stimulatory molecules and are impaired as antigen presenting cells. American Journal of Pathology, 150:641-651 (1997).
Panje W R. Regression of head and neck carcinoma with a prostaglandin-synthesis inhibitor. Arch Otolaryngol 1981; 107: 658-63
Pearson and Choi, Expression of the human b-amyloid precursor protein gene from a yeast artificial chromosome in transgenic mice. Proc. Natl. Acad. Sci. USA, 90:10578-82 (1993).
Randolph G. Dendritic cell migration to lymph nodes: cytokines, chemokines and lipid mediators. Seminars in Immunology, 13:267 (2001).
Ridgway D. The First 1000 Dendritic Cell Vaccines. Cancer Investigation, 21:873-886 (2003).
Roitt I, Brostoff J, Male D. Immunology, JB Lippincoft Co, Phila, Pa., (1989).
Romani N, Reider D, Heuer M, Ebner S, Kampgen E, Eibl B, Niederwieser D, and Schuler G. Generation of mature dendritic cells from blood. An improved method with regard to clinical applicability. Journal of Immunological Methods, 196:137-151 (1996).
Rogers P, and Croft M. CD28, Ox-40, LFA-1 and CD-4 modulation of Th1/Th2 differentiation is directly dependent on the dose of the antigen. The Journal of Immunology, 164:2955-2963 (2000).
Rosenberg S A, Yang J C, and Restifo N P. Cancer immunotherapy: moving beyond current vaccines. Natural Medicine, 10:909-915 (2004).
Rothstein, “Targeting, disruption, replacement, and allele rescue: integrative DNA transformation in yeast” in Methods in Enzymology, Vol. 194, “Guide to Yeast Genetics and Molecular Biology”, eds. C. Guthrie and G. Fink, Academic Press, Inc., Chap. 19, pp. 281-301 (1991).
Saha A, Hadden E M, Hadden J W. Zinc induces thymulin secretion from human thymic epithelial cells in vitro and augments splenocytes and thymocyte response in vivo. Int'l J Immunopharmacol 17:729-734 (1995).
Sahin U, Tureci O, Pfreundschuh. Serological identification of human tumor antigens. Curr Opin Immunol 9:709-715 (1997).
Saito H, Tsujitani S,lkeguchi M, Maeta M, and Kaibara N. Relationship between the expression of vascular endothelial growth factor and the density of dendritic cells in gastric adenocarcinoma tissue. British Journal of Cancer, 78:1573-1579 (1998).
Saito T, Kuss I, Dworacki G, Gooding W, Johnson J, and Whiteside T. Spontaneous ex Vivi Apoptosis of Peripheral Blood Mononuclear Cells in Patients with Head and Neck Cancer. Clinical Cancer Research, 5:1263-1273 (1999).
Sallusto F, Cella M, Danieli C, and Lanzavecchia A. Dendritic cells use macropinocytosis and the mannose receptor to concentrate macromolecules in the major histocompatibility complex class II compartment: down regulation by cytokines and bacterial products. Journal of Experimental Medicine, 182:389 (1995).
Sallusto F, and Lanzavecchia A. Efficient presentation of soluble antigen by cultured human dendritic cells is maintained by granulocyte/macrophage colony-stimulating factor plus interleukin 4 and down regulated by tumor necrosis factor α. Journal of Experimental Medicine, 179:1109 (1994).
Sanda M G, Smith D C, Charles L G. Recombinant vaccinia-PSA (Prostvac) can include a prostate-specific immune response in androgen-modulated human prostate cancer. Urology 52:2 (1999).
Schedl et al., “A yeast artificial chromosome covering the tyrosinase gene confers copy number-dependent expression in transgenic mice”, Nature, Vol. 362, pp. 258-261 (1993).
Schnurr M, Then F, Galambos P, Scholz C, Siegmund B, Endres S, and Eigier A. Extracellular ATP and TNF-α synergize in the activation and maturation of human dendritic cells. Journal of Immunology, 165:4704 (2000).
Schuler-Thurner B, Schultz E S, Berger T G, Weinlich G, Ebner S, Woerl P, Bender A, Feuerstein B, Fritsch P O, Romani N, and Schuler G. Rapid Induction of Tumor-specific Type 1 T Helper Cells in Metastatic Melanoma Patients by Vaccination with Mature, Cryopreserved, Peptide-loaded Monocyte-derived Dendritic Cells. J. Exp. Med., 195:1279-1288 (2002).
Smith B, Smith G, Carter D, Sasaki C, and Haffty B. Prognostic Significance of Vascular Endothelial Growth Factor Protein Levels in Oral and Oropharyngeal Squamous Cell Carcinoma. Journal of Clinical Oncology, 18:2048-2052 (2000).
Sorg R, Ozcan Z, Brefort T, Fischer J, Ackermann R, Muller M, and Wernet P. Clinical-Scale Generation of Dendritic Cells in a Closed System. Journal of Immunotherapy, 26:374-384 (2003).
Sozzani S, Allavena P, D'Amico G, Luini W, Bianchi G, Kataura M, Imai T, Yoshie O, Bonecchi R, and Mantovani A. Differential regulation of chemokine receptors during dendritic cell maturation: a model for their trafficking properties. Journal of Immunology, 161,1083 (1998).
Sprent, et al, Science, Vol. 293, 245-248 (2001).
Steinman R M. The dendritic cell system and its role in immunogenicity. Annual Review of Immunology, 9:271-296 (1991).
Steinman R, and Nussenzweig M. Avoiding horro autotoxicus: The importance of dendritic cells in peripheral T cell tolerance. Proceedings of the National Academy of Science USA, 99:351-358 (2002).
Strauss et al., “Germ line transmission of a yeast artificial chromosome spanning the murine al (I) collagen locus”, Science, Vol. 259, pp. 1904-1907 (1993).
Tagawa M. Cytokine therapy for cancer. Current Pharmaceut Design 6(6):681-699 (2000).
Takahashi A, Kono K, Ichihara F, Sugai H, Fujii H, and Matsumoto Y. Vascular Endothelial Growth Factor Inhibits Maturation of Dendritic Cells Induced by Lipopolysaccharide, but not by Proinflammatory Cytokines. Cancer Immunology and Immunotherapy, 53:543-550 (2004).
Tas M, Simons P, Balm F, and Drexhage H. Depressed monocyte polarization and clustering of dendritic cells in patients with head and neck cancer: in vitro restoration of this immunosuppression by thymic hormones. Cancer Immunology and Immunotherapy, 36:108-114 (1993).
Thurnher M, Radmayr C, Ramoner R, Ebner S, Bock G, Klocker H, Romani N, and Bartsch G. Human renal-cell carcinoma tissue contains dendritic cells. International Journal of Cancer, 68:1-7 (1996).
Thurnher B, Haendle I, Roder C, Dieckmann D, Keikavoussi P, Jonuleit H, Bender A, Maczek C, Schreiner D, von den Driesch P, Brocker E B, Steinman R M, Enk A, Kampgen E, and Schuler G. Vaccination with Mage-3A1 Peptide-pulsed Mature, Monocyte-derived Dendritic Cells Expands Specific Cytotoxic T Cells and Induces Regression of Some Metastases in Advanced Stage IV Melanoma. Journal of Experimental Medicine, 190:1669-1678 (1999).
Valente G, DeStefani A, Jemma C, Giovarelli M, Geuna N, Cortesina G, Forni G, Palestro G. Infiltrating leukocyte populations and T-lymphocyte subsets in head and neck squamous cell carcinomas from patients receiving perilymphatic injections of recombinant interleukin-2. A pathologic and immunophenotypic study. Modern Pathol 3(6):702-708 (1990).
Valente G, DeStefani A, Jemma C. Infiltrating leukocyte populations and T lymphocyte subsets in H&NSCC from patients receiving perilymphatic injections of rIL-2. Mod Pathol 1990; 3: 702-708
Van der Eynde B, Van der Bruggen, T cell defined tumor antigens. Curr Opin Immunol 9:684-693 (1997).
Verastegui E, Barrera J L, Zinzer J, del Rio R, Meneses A, de la Garza J, Hadden J W. A natural cytokine mixture (IRX-2) and interference with immune suppression induce immune mobilization and regression of head and neck cancer. Int'l J Immunopharmacol 11/12:619-627 (1997).
Verastegui E, Hadden EM, Barrera J, Meneses A, Hadden J W. A natural cytokine mixture (IRX-2) and interference with immune suppression induce immune regression and improved survival in head and neck cancer. Int'l. Journal of Immunorehabilitation; 1999; 12: 5-11.
Wang R F, Rosenberg S A. Human tumor antigens for cancer vaccine development. Immunologic Reviews 170:85-100 (1999).
Weber J. Tumor vaccines. Medscape Anthology 3:2 (2000).
Whiteside, et al, Cancer Res. 53:5654-5662, (1993).
Whiteside T, Stanson J, Shurin M, and Ferrone S. Antigen-Processing Machinery in Human Dendritic Cells: Up-Regulation by Maturation and Down-Regulation by Tumor Cells. The Journal of Immunology, 173:1526-1534 (2004).
Whiteside T. Immunobiology and immunotherapy of head and neck cancer. Current Oncology Report, 2001:346-355 (2001).
Wolf et al, Arch. Oto. Laryngol. 111:716-725 (1985).
Zhou L, and Tedder T. CD14⁺ blood monocytes can differentiate into functionally mature CD83⁺ dendritic cells. Proceedings of the National Academy of Science USA, 93:2588 (1996).

What is claimed is:

1. A diagnostic skin test for predicting treatment outcome, consisting essentially of an effective amount of a natural cytokine mixture (NCM).

2. The diagnostic skin test of claim 1, wherein said NCM includes cytokines IL-1, IL-2, IL-6, IL-8, IFN-γ, and TNF-α.

3. The diagnostic skin test of claim 2, wherein said NCM further includes cytokines IL-12, GM-CSF, and G-CSF.

4. The diagnostic skin test of claim 3, wherein said cytokines are chosen from the group consisting of recombinant, natural, or pegylated cytokines.

5. The diagnostic skin test of claim 4, wherein said NCM includes 4-50 units of IL-2.

6. The diagnostic skin test of claim 1, wherein the NCM is provided in a pharmaceutically acceptable carrier.

7. A kit for performing a skin test consisting essentially of an effective amount of a natural cytokine mixture (NCM).

8. The kit of claim 7, wherein said NCM includes cytokines IL-1, IL-2, IL-6, IL-8, IFN-γ, and TNF-α.

9. The kit of claim 8, wherein said NCM further includes cytokines IL-12, GM-CSF, and G-CSF.

10. The kit of claim 9, wherein said cytokines are chosen from the group consisting of recombinant, natural, or pegylated cytokines.

11. The kit of claim 10, wherein said NCM includes 4-50 units of IL-2.

12. The kit of claim 7, wherein said NCM is provided in a pharmaceutically acceptable carrier.

13. A method of performing a skin test on a patient, consisting essentially of the steps of administering an effective amount of a natural cytokine mixture (NCM) to skin, analyzing results of the skin test, and predicting a treatment outcome.

14. The method of claim 13, wherein the NCM includes 4-50 units of IL-2.

15. The method of claim 13, wherein the patient is a cancer patient.

16. The method of claim 13, wherein said administering step includes administering the NCM intradermally.

17. The method of claim 13, wherein said analyzing step includes reading the test from 6 to 48 hours after administration.

18. The method of claim 17, wherein said analyzing step is further defined as reading the test 24 hours after administration.

19. The method of claim 13, wherein said predicting step is further defined as predicting a treatment outcome chosen from the group consisting of overall survival of the patient, response to immunotherapy, response to surgery, response to radiotherapy, time to recurrence, and time to death.

20. The method of claim 13, wherein a negative response to the skin test predicts a negative treatment outcome.

21. The method of claim 13, wherein a negative response to the skin test predicts at least one defect in monocyte function.

22. The method of claim 13, wherein a positive response to the skin test predicts a positive treatment outcome.

23. A diagnostic skin test for predicting treatment outcome, consisting essentially of an effective amount of a T lymphocyte mitogen of muromonab-CD3.

24. The diagnostic skin test of claim 23, including 0.1 to 100 ng of muromonab-CD3.

25. The diagnostic skin test of claim 23, wherein the muromonab-CD3 is provided in a pharmaceutically acceptable carrier.

26. A kit for performing a skin test consisting essentially of an effective amount of a T lymphocyte mitogen of muromonab-CD3.

27. The kit of claim 26, including 0.1 to 100 ng of muromonab-CD3.

28. The kit of claim 27, wherein said muromonab-CD3 is provided in a pharmaceutically acceptable carrier.

29. A method of performing a skin test on a patient, consisting essentially of the steps of administering an effective amount of a T lymphocyte mitogen of muromonab-CD3 to skin, analyzing results of the skin test, and predicting a treatment outcome.

30. The method of claim 29, including 0.1 to 100 ng of muromonab-CD3.

31. The method of claim 29, wherein the patient is a cancer patient.

32. The method of claim 29, wherein said administering step includes administering the muromonab-CD3 intradermally.

33. The method of claim 29, wherein said analyzing step includes reading the test from 6 to 48 hours after administration.

34. The method of claim 33, wherein said analyzing step is further defined as reading the test 24 hours after administration.

35. The method of claim 29, wherein said predicting step is further defined as predicting a treatment outcome chosen from the group consisting of overall survival of the patient, response to immunotherapy, response to surgery, response to radiotherapy, time to recurrence, and time to death.

36. The method of claim 29, wherein a negative response to the skin test predicts a negative treatment outcome.

37. The method of claim 29, wherein a negative response to the skin test predicts at least one defect in T lymphocyte function.

38. The method of claim 29, wherein a positive response to the skin test predicts a positive treatment outcome.

39. A method of detecting defects in monocyte function, including the steps of administering an effective amount of a natural cytokine mixture (NCM) to skin, analyzing results of the skin test, and detecting at least one defect in monocyte function.

40. A method of detecting defects in T lymphocyte function, including the steps of administering an effective amount of a T lymphocyte mitogen of muromonab-CD3 to skin, analyzing results of the skin test, and detecting at least one defect in monocyte function.

41. Means for indicating a functioning efferent limb of an immune system, consisting essentially of a diagnostic skin test including an effective amount of a natural cytokine mixture (NCM).

42. Means for indicating a functioning afferent limb of an immune system, consisting essentially of a diagnostic skin test including an effective amount of a T lymphocyte mitogen of muromonab-CD3.
